(19) United States
(12) Patent Application Publication
(10) Pub. No.: US 2009/0263744 A1
(43) Pub. Date: Oct. 22, 2009

(54) ETHYLENICALLY UNSATURATED COMPOUND, LIGHT SENSITIVE COMPOSITION, LIGHT SENSITIVE PLANOGRAPHIC PRINTING PLATE MATERIAL AND PRINTING PROCESS EMPLOYING THE SAME

(76) Inventor: Takaaki Kuroki, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/097,695

(22) PCT Filed: Dec. 7, 2006
(86) PCT No.: PCT/JP2006/324445

§ 371 Date: Jun. 16, 2008

(30) Foreign Application Priority Data
Dec. 19, 2005 (JP) 2005-364550
Mar. 30, 2006 (JP) 2006-093450

(51) Int. Cl. G03F 7/004; C07C 271/08; B41F 7/00

(52) U.S. Cl. 430/281.1; 560/158; 101/130

(57) ABSTRACT

The present invention provides a novel ethylenically unsaturated compound which is highly sensitive to scanning exposure due to an ultraviolet to near-infrared laser and is cured to give good physical properties; a light sensitive composition providing good developability and high sensitivity and forming a high strength layer; a planographic printing plate material having advantages that good developability, high sensitivity and high strength layer are obtained, particularly a planographic printing plate material requiring no developing machine which are mounted on a printing press without any development, followed by printing and which provides excellent developability on a printing press and printing durability; and a printing process. The ethylenically unsaturated compound has in the molecule a photo-oxidation group and a polymerizable ethylenically unsaturated bond, and has a predetermined solubility in water or an aqueous alkali solution. The planographic printing plate material is characterized in that it comprises a support and provided thereon, a light sensitive layer containing the ethylenically unsaturated compound, a polyhalogen compound as a photopolymerization initiator, a water-soluble polymer binder as a polymer binder and an infrared absorbing agent.

### FIELD OF THE INVENTION

The present invention relates to a novel ethylenically unsaturated compound, a light sensitive composition containing the same, a light sensitive planographic printing plate material and a printing process employing the same, and to a light sensitive planographic printing plate material for so-called a computer-to-plate system (hereinafter referred to as CTP system), and a printing process employing the same.

Particularly, the present invention relates to a planographic printing plate material which is capable of forming an image by laser scanning exposure and which is mounted on a printing press immediately after exposure, followed by printing, and a printing process.

### TECHNICAL BACKGROUND

A light sensitive composition is widely used in imaging fields, and has been long studied. However, its industrial growth causes requirements of the market such as high sensitivity, high resolution, high strength, and ease of handling, and the technical innovation in the imaging fields does not cease.

The light sensitive composition is applied to the imaging fields such as phototyping, holography or color hard copy; electronic material manufacturing fields such as a photoresist or a color filter; and photocuring resin fields such as ink, coatings or adhesives, and demands for a novel high-function materials are strong. One of such a field is a printing material field.

Presently, digital technique electronically processing, storing and outputting image information employing a computer has spread. In a plate making system of a planographic printing plate for off-set printing, a computer-to-plate system (hereinafter referred to as CTP system), which writes a digital image directly on a light sensitive planographic printing plate material employing a laser, has been developed and put into practical use.

In a planographic printing plate material field, as image formation technique proceeds, a light sensitive planographic printing plate material which is highly sensitive to a relatively inexpensive and low output power laser has been required. For example, a light sensitive planographic printing plate material which can be applied to an output equipments employing an argon laser, a helium-neon laser, a red LED and the like is actively studied.

As a typical laser, a semiconductor laser emitting a continuous-wave laser in the wavelength region of 350 to 450 nm and using InGaN based materials has now entered the stage of practical use. A scanning exposure system using such a light source of a short wavelength has advantages that a semiconductor laser can be manufactured at a relatively low cost in terms of structure and an economical system with a sufficient output can be realized. Such a light source can be used for a planographic printing plate material which are sensitive to the shorter wavelength region and workable under a lighter safe light, compared to a conventional system using a FD-YAG laser or an Ar laser. The semiconductor laser is one of the preferred embodiments.

On the other hand, a 700-1300 nm near-infrared laser source is relatively inexpensive, and a light sensitive planographic printing plate material applied to light having such a wavelength is noted.

As photopolymerizable compositions having sensitivity in the visible to near-infrared range, there are a planographic printing plate material comprising a radically polymerizable ethylenically unsaturated compound, a sensitizing dye having absorption maximum in the range of from 400 to 500 nm and a polymerization initiator disclosed in Japanese Patent O.P.I. Publication No. 9-134007, a combination of an organic boron anion and a dye disclosed in Japanese Patent O.P.I. Publication Nos. 62-143044, 62-150242, 5-5988, 5-194619, 5-197069, and 2000-98603, a combination of a dye and an S-triazine disclosed in Japanese Patent O.P.I. Publication Nos. 4-31863 and 6-43633, a combination of a resol resin, a novolak resin, an infrared absorbing agent and a photo-acid-generating agent disclosed in Japanese Patent O.P.I. Publication Nos. 7-20629 and 7-271029, and a combination of a specific polymer, a photo-acid-generating agent and a near-infrared sensitizing dye disclosed in Japanese Patent O.P.I. Publication Nos. 11-212252 and 11-231535. A light sensitive composition containing a polymer having a vinyl group-substituted phenyl group is disclosed in Japanese Patent Publication No. 6-105353 and Japanese Patent O.P.I. Publication No. 2001-290271 (Patent Documents 1 and 2).

However, a planographic printing plate material is not still obtained which provides sufficient sensitivity to laser scanning exposure, high printing durability and good developability with less fatigue of developer.

The binder polymer used in the light sensitive layer of the examples described above is insoluble in water, and the light sensitive layer requires liquid development employing an aqueous alkali solution containing a strong alkali agent such as sodium hydroxide, potassium hydroxide or sodium silicate in order to form an image.

A planographic printing plate material which is water-developable or does not require development is desired on the marked in view of stability of printing, ease of control or environmental concern.

For example, a processless planographic printing plate material requiring no development, which ablates a polymer binder due to a high output power near-infrared semiconductor laser, is proposed. For example, a planographic printing plate manufacturing method is disclosed in Japanese Patent O.P.I. Publication No. 8-48018 which removes ink-receptive polymer by ablation and exposes a surface of a hydrophilic support by a washing agent, however, this produces ablation residue called debris, which may result in contamination of an optical equipment used or background contamination of a printing plate.

Further, a water-developable thermosensitive planographic printing plate material comprising a thermosensitive layer containing a thermoplastic polymer particles is disclosed in Japanese Patent O.P.I. Publication Nos. 9-171250, 10-186646 and 11-265062 and U.S. Pat. No. 6,001,536 which is exposed to the high output power near-infrared semiconductor laser to fuse the polymer particles by heat generated at exposed portions, resulting in water insolubility of the thermosensitive layer at exposed portions. A thermosensitive planographic printing plate material is disclosed which employs a water-soluble polymer having a specific chemical structure (see Patent Document 3).

These are preferred ones which are mounted on a printing press without any development, followed by printing, however, there are problems in that they are less sensitive to laser, insufficient in printing durability and is likely to produce background contamination, which do not provide satisfactory properties.

A light sensitive composition which excels in sensitivity and developability has been sought in the imaging fields (see for example Patent Document 4), and its application to the imaging fields such as phototyping, holography or color hard copy; electronic material manufacturing fields such as a photoresist; and photocuring resin fields such as ink, coatings or adhesives is desired.

In view of the above technical background, an excellent ethylenically unsaturated compound is strongly desired as a main component constituting the light sensitive composition which excels in sensitivity and developability.

### Patent Document 1: Japanese Patent Publication No. 6-105353

### Patent Document 2: Japanese Patent O.P.I. Publication No. 2001-290271

### Patent Document 3: Japanese Patent O.P.I. Publication No. 2003-215801

### Patent Document 4: Japanese Patent O.P.I. Publication No. 2000-258910

### DISCLOSURE OF THE INVENTION

### Problems to be Solved by the Invention

The present invention has bee made in view of the above. An object of the invention is to provide a novel ethylenically unsaturated compound which is highly sensitive to scanning exposure due to an ultraviolet to near-infrared laser and is cured to give excellent physical properties; a light sensitive composition giving good developability and high sensitivity and forming a high strength layer; and a planographic printing plate material having advantages that good developability, high sensitivity and high strength layer are obtained, particularly a planographic printing plate material requiring no developing machine, which are mounted on a printing press without any development, followed by printing; and a printing process.

Another object of the invention is to provide a planographic printing plate material requiring no developing machine, which is highly sensitive to scanning exposure due to an infrared to near-infrared laser and which is mounted on a printing press without any development, followed by printing, particularly a planographic printing plate material which excels in on-press development property and printing durability which are opposing properties, and a printing process.

### Means for Solving the Above Problems

The above object of the invention can be attained by any one of the following constitutions.

1. An ethylenically unsaturated compound having in the molecule a photo-oxidation group and a polymerizable ethylenically unsaturated bond, wherein the ethylenically unsaturated compound dissolves in an amount of not less than 1% by weight in a 25° C. KOH aqueous solution having a pH of 12.5.

2. An ethylenically unsaturated compound having in the molecule a photo-oxidation group and a polymerizable ethylenically unsaturated bond, wherein the ethylenically unsaturated compound dissolves in an amount of not less than 1% by weight in a 25° C. pure water.

3. The ethylenically unsaturated compound of item 1 or 2 above, wherein the ethylenically unsaturated compound is a polymerizable ethylenically unsaturated compound having in the molecule an amido bond and a secondary or tertiary amino group.

4. The ethylenically unsaturated compound of any one of items 1 through 3 above, wherein the ethylenically unsaturated compound is a polymerizable ethylenically unsaturated compound further having in the molecule an alkyleneoxy structure.

5. A light sensitive composition containing at least (1) a polymerizable ethylenically unsaturated compound and (2) a photopolymerization initiator, the polymerizable ethylenically unsaturated compound being the ethylenically unsaturated compound of any one of items 1 through 4 above.

6. A light sensitive planographic printing plate material comprising a support and provided thereon, a light sensitive layer containing at least (1) a polymerizable ethylenically unsaturated compound, (2) a photopolymerization initiator and (3) a polymer binder, wherein the polymerizable ethylenically unsaturated compound is the ethylenically unsaturated compound of any one of items 1 through 4 above.

7. A light sensitive planographic printing plate material comprising a support and provided thereon, an infrared ray-polymerizable light sensitive layer containing at least (1) a polymerizable ethylenically unsaturated compound, (2) a photopolymerization initiator, (3) a polymer binder, and (4) an infrared absorbing agent, wherein the light sensitive layer contains the ethylenically unsaturated compound of any one of items 1 through 4 above as the polymerizable ethylenically unsaturated compound, a polyhalogen compound as the photopolymerization initiator, and a water-soluble polymer binder as the polymer binder.

8. A light sensitive planographic printing plate material comprising a support and provided thereon, a light sensitive layer containing at least (1) a polymerizable ethylenically unsaturated compound, (2) a photopolymerization initiator, (3) a polymer binder, and (4) an infrared absorbing agent, wherein the light sensitive layer contains a polymerizable ethylenically unsaturated compound having in the molecule a photo-oxidation group as the polymerizable ethylenically unsaturated compound, a polyhalogen compound as the photopolymerization initiator, and a water-soluble polymer binder as the polymer binder.

9. A light sensitive planographic printing plate material comprising a support and provided thereon, a light sensitive layer containing at least (1) a polymerizable ethylenically unsaturated compound, (2) a photopolymerization initiator, (3) a polymer binder, and (4) an infrared absorbing agent, wherein the light sensitive layer contains a polymerizable ethylenically unsaturated compound having in the molecule an amido bond and a secondary or tertiary amino group as the polymerizable ethylenically unsaturated compound, a polyhalogen compound as the photopolymerization initiator, and a water-soluble polymer binder as the polymer binder.

10. The light sensitive planographic printing plate material of item 8 or 9 above, wherein the polymerizable ethylenically unsaturated compound dissolves in an amount of not less than 1 by weight in a 25° C. pure water.

11. A printing process comprising the steps of mounting the light sensitive planographic printing plate material of any one of items 6 through 10 above without carrying out development, followed by printing.

### EFFECTS OF THE INVENTION

The present invention can provide a novel ethylenically unsaturated compound which is highly sensitive to scanning exposure due to an ultraviolet to near-infrared laser and is cured to give excellent physical properties; a light sensitive composition providing good developability and high sensitivity and forming a high strength layer; a planographic printing plate material having advantages that good developability, high sensitivity and high strength layer are obtained, particularly a planographic printing plate material requiring no developing machine, which are mounted on a printing press without any development, followed by printing; and a printing process.

The present invention can provide a planographic printing plate material requiring no developing machine, which is highly sensitive to scanning exposure due to an infrared to near-infrared laser and which is mounted on a printing press without any development, followed by printing, and particularly to a planographic printing plate material which excels in on-press developability and printing durability which are opposing properties, and a printing process.

### PREFERRED EMBODIMENT OF THE INVENTION

The present invention and the constituents will be explained in detail below.

### (Polymerizable Ethylenically Unsaturated Compound)

The present invention is characterized in that a polymerizable ethylenically unsaturated compound having in the molecule a photo-oxidation group is employed as a polymerizable ethylenically unsaturated compound.

As the photo-oxidation group, there are a thio group, a thioether group, a ureido group, an amino group, and an enol group. Examples thereof include a triethanolamino group, a triphenylamino group, a thioureido group, an imidazolyl group, an oxazolyl group, a thiazolyl group, an acetylacetonyl group, an N-phenylglycine residue, and an ascorbic acid residue. Among these, a secondary or tertiary amino group is preferred.

In the invention, a polymerizable ethylenically unsaturated compound having in the molecule an amide bond and a secondary or tertiary amino group is preferred as the polymerizable ethylenically unsaturated compound.

The polymerizable ethylenically unsaturated compound having in the molecule an amide bond and a secondary or tertiary amino group forms a layer with high adhesion and high strength. High adhesion and high strength of the layer result from dispersion force or intermolecular force of the amide bond, and from photo-oxidation of the secondary or tertiary amino group, which increases cross-linking density on exposure.

Examples of the compound having a photo-oxidation group include compounds described in European patent publication Nos. 287,818, 353,389 and 364,735. Among them, preferred are compounds having a tertiary amino group and a urethane group and compounds having a ureido group and a urethane group.

As compounds having at least one photo-oxidation group and at least one urethane group, there are those disclosed in Japanese Patent O.P.I. Publication No. 63-260909, Japanese Patent No. 2669849, and Japanese Patent O.P.I. Publication Nos. 6-35189 and 2001-125255.

The most preferred compound, a polymerizable ethylenically unsaturated compound containing in the molecule an amide bond and a secondary or tertiary amino group will be explained in detail below.

The polymerizable ethylenically unsaturated compound containing in the molecule an amide bond and a secondary or tertiary amino group is a compound satisfying the following requirement 1) or 2).

1) Not less than 10 by weight of the compound dissolves in a 25° C. KOH aqueous solution having a pH of 12.5.
2) Not less than 1% by weight of the compound dissolves in 25° C. pure water.

In the invention, in item 1) or 2), the polymerizable ethylenically unsaturated compound dissolves in an amount of preferably not less than 3% by weight, more preferably not less than 5% by weight, and still more preferably not less than 10% by weight.

Herein, “dissolve” implies that no precipitations or oily separated components are visually observed under the following experimental conditions. Solubility in a 25° C. KOH aqueous solution having a pH of 12.5 or 25° C. pure water is represented by a value obtained by the following measuring method.

An ethylenically unsaturated compound is added to 20 ml of the solution or pure water described above at 25° C. in a 100 ml conical flask, stirred with a stirrer having a 1 cm long stirrer piece at a rate of 300 rpm for one hour, then allowed to stand for five minutes. The resulting solution was observed, and the amount of the compound, at which no precipitations or oily separated components are visually observed in the solution, is defined as solubility, which is represented by % by weight.

As a compound having a property described above, there is, for example, a reaction product of three components, a polyhydric alcohol (a) having a secondary or tertiary amino group in the molecule, a polyisocyanate (b), and a compound (c) having both a hydroxyl group and an addition polymerizable ethylenically double bond in the molecule.

A polyhydric alcohol unit having no amino group such as glycols in addition to the three components above is preferably incorporated into the reaction product. The content by mole of a compound having a double bond in the reaction product is preferably less than 40%, more preferably less than 30%, and still more preferably less than 25%.

The component of the polymerizable ethylenically unsaturated compound in the invention having an amido group and a secondary or tertiary amino group in the molecule will be explained in detail below.

### [Polyhydric Alcohol (a) Having a Tertiary Amino Group in the Molecule]

Examples of the polyhydric alcohol (a) having a secondary or tertiary amino group herein referred to include triethanolamine, N-methyldiethanolamine, N-ethyldiethanolamine, N-ethyldiethanolamine, N-n-butyldiethanolamine, N-tert-butyldiethanolamine, N,N-di(hydroxyethyl)aniline, N,N,N′,N′-tetra-2-hydroxypropylethylenediamine, p-tolyldiethanolamine, N,N, N′, N′-tetra-2-hydroxyethylethylenediamine, N,N-bis(2-hydroxypropyl)aniline, allyldiethanolamine, 3-dimethylamino-1,2-propane diol, 3-diethylamino-1,2-propane diol, N,N-di(n-propylamino)-2,3-propane diol, N,N-di(iso-propylamino)-2,3-propane diol, and 3-(N-methyl-N-benzylamino)-1,2-propane diol, but the invention is not specifically limited thereto.

[Polyisocyanate (b)]

The polyisocyanate (b) refers to a compound having in the molecule two or more of an isocyanate group, and may be a polyol, biuret or isocyanuric acid adduct of a polyisocyanate.

Examples of the diisocyanate include butane-1,4-diisocyanate, hexane-1,6-diisocyanate, 2-methylpentane-1,5-diisocyanate, octane-1,8-diisocyanate, 1,3-diisocyanatomethylcyclohexanone, 2,2,4-trimethylhexane-1,6-diisocyanate, isophorone diisocyanate, 1,2-phenylene diisocyanate, 1,3-phenylene diisocyanate, 1,4-phenylene diisocyanate, tolylene-2,4-diisocyanate, tolylene-2,5-diisocyanate, tolylene-2,6-diisocyanate, 1,3-di(isocyanatomethyl)benzene, and 1,3-bis(1-isocyanato-1-methylethyl)benzene.

There are, further, diisocyanates such as norbornene diisocyanate, m-phenylene diisocyanate, p-phenylene diisocyanate, 2,6-tolylene diisocyanate, 2,6-tolylene diisocyanate, 2,4-tolylene diisocyanate, naphthalene-1,4-diisocyanate, 4,4′-diphenylmethane diisocyanate, polymeric diphenylmethane diisocyanate, dicyclohexylmethane-4,4′-diisocyanate, 5-isocyanate-1-(isocyanatemethyl=1,3,3-trimethylcyclohexane, 3,3′-dimethyldiphenylmethane-4,4′-diisocyanate, xylylene-1,4-diisocyanate, 4,4′-diphenylpropane diisocyanate, trimethylene diisocyanate, hexamethylene diisocyanate, propylene-1,2-diisocyanate, butylene-1,2-diisocyanate, cyclohexylene-1,2-diisocyanate, and cyclohexylene-1,4-diisocyanate.

There are, further, triisocyanates such as 4,4′,4″-triphenylmethane triisocyanate and toluene-2,4,6-triisocyanate, and tetraisocyanates such as 4,4′-dimethyldiphenylmethane-2,2′,5,5′-tetraisocyanate. As the adduct of polyisocyanates and polyols, there are, for example, a trimethylolpropane adduct of hexamethylene diisocyanate, a trimethylolpropane adduct of 2,4-tolylene diisocyanate, a trimethylolpropane adduct of xylylene diisocyanate, and a hexanetriol adduct of tolylene diisocyanate. The polyisocyanate is not limited to the compound as described above, and several kinds thereof may be used in combination.

### [Compound (c) Having Both a Hydroxyl Group and an Addition Polymerizable Ethylenically Double Bond in the Molecule]

Examples of the compound having both a hydroxyl group and an addition polymerizable ethylenically double bond in the molecule is not specifically limited, but include 2-hydroxyethyl methacrylate, 2-hydroxyethyl acrylate, 4-hydroxybutyl acrylate, 2-hydroxypropylene-1,3-dimethacrylate, and 2-hydroxypropylene-1-methacrylate-3-acrylate.

The reaction product can be synthesized according to the same method as a conventional method in which a urethaneacrylate compound is ordinarily synthesized employing a diol, a diisocyanate and an acrylate having a hydroxyl group.

Examples of the reaction product of a polyhydric alcohol having a tertiary amino group in the molecule, a diisocyanate, and a compound having a hydroxyl group and an addition polymerizable ethylenically double bond in the molecule will be listed below.

M-1: A reaction product of triethanolamine (1 mole), hexane-1,6-diisocyanate (3 moles), and 2-hydroxyethyl methacrylate (3 moles)
M-2: A reaction product of triethanolamine (1 mole), isophorone diisocyanate (3 moles), and 2-hydroxyethyl methacrylate (3 moles)
M-3: A reaction product of N-n-butyldiethanolamine (1 mole), 1,3-bis(1-cyanato-1-methylethyl)benzene (2 moles), and 2-hydroxypropylene-1-methacrylate-3-acrylate (2 moles)
M-4: A reaction product of N-n-butyldiethanolamine (1 mole), 1,3-di(cyanatomethyl)benzene (2 moles), and 2-hydroxypropylene-1-methacrylate-3-acrylate (2 moles)
M-5: A reaction product of N-methyldiethanolamine (1 mole), tolylene-2,4-diisocyanate (2 moles), and 2-hydroxypropylene-1,3-dimethacrylate (2 moles)

Of reaction products comprised of combinations of these components is preferred one satisfying the following requirement 1) or 2).

1) Not less than 1% by weight of the reaction product dissolves in a 25° C. KOH aqueous solution having a pH of 12.5.

2) Not less than 1% by weight of the reaction product dissolves in 25° C. pure water.

In the invention, in item 1) or 2) above, the reaction product dissolves in an amount of preferably not less than 3% by weight, more preferably not less than 5% by weight, and still more preferably not less than 10% by weight.

A compound having a property as described above makes it possible to attain the object of the invention.

In the invention, a compound having such a specific property of known compounds can be preferably used, and a reaction product into which a polyhydric alcohol unit having no amino group (as the fourth component) is incorporated in addition to the three components above is more preferred. Examples of the polyhydric alcohol having no amino group will be explained in detail below.

[Polyhydric Alcohol (d) having No Amino Group]

Examples of the polyhydric alcohol (d) having no amino group include polyhydric alcohols such as ethylene glycol, 1,2- or 1,3-propylene glycol, butylene glycol, diethylene glycol, triethylene glycol, tetraethylene glycol, dipropylene glycol, 1,3- or 1,4-butane diol, 1,5-pentane diol, 1,6-hexane diol, neopentyl glycol, 2-methylpentane diol, hydrogenated bisphenol A, polyethylene glycol, polypropylene glycol, polybutylene glycol, polytetramethylene glycol, polycaprolactone, trimethylolethane, trimethylolpropane, polytrimethylol-propane, pentaerythritol, polypentaerythritol, sorbitol, mannitol, glycerin and polyglycerin; polyesterpolyols such as reaction products of the polyhydric alcohols with polybasic acids such as maleic anhydride, maleic acid, fumaric acid, itaconic anhydride, itaconic acid, adipic acid, phthalic acid, phthalic anhydride, terephthalic acid, and isophthalic acid; caprolactone-modified polyols; polyolefin type polyols; polycarbonate type polyols; and polybutadiene type polyols. These can be used in combination with hydroxy fatty acid esters above.

Other examples of the polyhydric alcohol (d) include aliphatic polyhydric alcohols such as 1,3-propanediol, 1,7-heptanediol, 1,8-octanediol, 2,3-dihydroxybutane, 1,2-dihydroxybutane, 1,3-dihydroxybutane, 2,2-dimethyl-1,3-propanediol, 2,4-pentanediol, 2,5-pentanediol, 3-methyl-1,5-pentanediol, 1,4-cyclohaxanedimethanol, dihydroxycyclohexane, 1,2,6-trihydroxycyclohexane, phenylethylene glycol, 1,1,1-trimethylolpropane, hexanetriol, pentaerythritol, and glycerin; condensation products of alkylene oxides with aromatic polyhydric alcohols such as 1,4-di(2-hydroxyethoxy)benzene and 1,3-bis(2-hydroxyethoxy)benzene, p-xylylene glycol, m-xylylene glycol, α,α′-dihydroxy-p-isopropylbenzene, 4,4′-dihydroxydiphenylmethane, 2-(p,p′-dihydroxydiphenylmethyl)benzyl alcohol, 4,4′-dihydroxydiphenylsulfone, 4,4′-dihydroxydiphenylsulfide, an ethylene oxide adduct of 4,4′-isopropylidenediphenol, and a propylene oxide adduct of 4,4′-isopropylidenediphenol.

Examples of the reaction product containing the fourth component will be listed below.

M-6: A reaction product of triethanolamine (1 mole), hexane-1,6-diisocyanate (3 moles), 2-hydroxyethyl methacrylate (3 moles) and diethylene glycol (3 moles)
M-7: A reaction product of triethanolamine (1 mole), isophorone diisocyanate (3 moles), 2-hydroxyethyl methacrylate (3 moles) and ethylene glycol (3 moles)
M-8: A reaction product of N-n-butyldiethanolamine (1 mole), 1,3-bis(1-cyanato-1-methylethyl)benzene (2 moles), 2-hydroxypropylene-1-methacrylate-3-acrylate (2 moles) and diethylene glycol (2 moles)
M-9: A reaction product of N-n-butyldiethanolamine (1 mole), 1,3-di(cyanatomethyl)benzene (2 moles), 2-hydroxypropylene-1-methacrylate-3-acrylate (2 moles) and diethylene glycol (2 moles)
M-10: A reaction product of N-methyldiethanolamine (1 mole), tolylene-2,4-diisocyanate (2 moles), 2-hydroxypropylene-1,3-dimethacrylate (2 moles) and diethylene glycol (2 moles)

It is preferred in the invention that the content by mole of a compound as the third component having both a hydroxyl group and an addition polymerizable ethylenically double bond in the molecule in the reaction product is less.

The content by mole of the third component in the reaction product is preferably less than 40%, more preferably less than 30%, and still more preferably less than 25%.

Examples of the reaction product containing the third component in less amount will be listed below.

M-11: A reaction product of N-n-butyldiethanolamine (2 moles) 1,3-bis(1-cyanato-1-methylethyl)benzene (3 moles) and 2-hydroxypropylene-1-methacrylate-3-acrylate (2 moles)
M-12: A reaction product of N-n-butyldiethanolamine (3 mole), hexamethylene diisocyanate (4 moles) and 2-hydroxypropylene-1-methacrylate-3-acrylate (2 moles)
M-13: A reaction product of N-methyldiethanolamine (2 mole), tolylene-2,4-diisocyanate (3 moles) and 2-hydroxypropylene-1,3-dimethacrylate (2 moles)

Of reaction products comprised of combinations of these components is preferred a compound satisfying any of the following requirements.

1) Not less than 1% by weight of the compound dissolves in a 25° C. KOH aqueous solution having a pH of 12.5.

2) Not less than 1% by weight of the compound dissolves in 25° C. pure water.

In the invention, in item 1) or 2) above, the compound dissolves in an amount of preferably not less than 3% by weight, more preferably not less than 5% by weight, and still more preferably not less than 10% by weight.

In order to obtain such a property, it is preferred that a polyol unit containing no amino group such as glycols is incorporated as the fourth component into the reaction product or the content by mole in the reaction product of a compound as the third component having both a hydroxyl group and an addition polymerizable ethylenically double bond in the molecule is less. It is especially preferred that a polyol unit containing no amino group such as glycols is incorporated as the fourth component into the reaction product.

The object of the invention can be attained by use of compounds having such a property.

The polymerizable ethylenically unsaturated compound in the invention is preferably one having in the molecule an alkyleneoxy structure.

The alkyleneoxy structure in the invention (alkylene oxide block) is preferably one containing a unit represented by formula —CₙH₂ₙ—O— (in which n is preferably an integer of from 2 to 4). The part —CₙH₂ₙ— may be straight chained or branched. Further, the alkylene part may have a substituent. Preferred examples of such a polymer include those disclosed in WO 99/21725 and Japanese Patent O.P.I. Publication No. 2004-131520.

As for example, a polyether polyol having in the molecule an alkyleneoxy structure, there is, for example, polyethylene glycol, polypropylene glycol, polytetramethylene glycol, polyhexamethylene glycol, polyheptamethylene glycol, or polydecamethylene glycol. Among these, polytetramethylene glycol is preferred.

As a polyether polyol having in the molecule an alkyleneoxy structure, those prepared by ring-opening copolymerization of two or more kinds of ionically polymerizable cyclic compounds are suitably used. Examples of the ionically polymerizable cyclic compounds include cyclic ethers such as ethylene oxide, propylene oxide, butane-1-oxide, isobutene oxide, 3,3-bischloromethyloxetane, tetrahydrofuran, 2-methyltetrahydrofuran, 3-methyltetrahydrofuran, dioxane, trioxane, tetraoxane, cyclohexane oxide, styrene oxide, epichlorohydrin, glycidyl methacrylate, allyl glycidyl ether, allyl glycidyl carbonate, butadiene monoxide, isoprene monoxide, vinyloxetane, vinyltetrahydrofuran, vinylcyclohexene oxide, phenyl glycidyl ether, butyl glycidyl ether and glycidyl benzoate.

Examples of those derived from two or more kinds of ionically polymerizable cyclic compounds include two-component copolymers obtained from a combination of tetrahydrofuran and propylene oxide, a combination of tetrahydrofuran and 2-methyltetrahydrofuran, a combination of tetrahydrofuran and 3-methyltetrahydrofuran, a combination of tetrahydrofuran and ethylene oxide and a combination of butene-1-oxide and ethylene oxide; and three-component copolymers obtained from a combination of tetrahydrofuran, butene oxide and ethylene oxide, and a combination of tetrahydrofuran, butene-1-oxide and ethylene oxide.

Polyether polyols obtained by ring-opening copolymerization of the ionically polymerizable cyclic compounds described above with cyclic imines such as ethylene imine, cyclic lactones such as β-propiolactone or glycolic acid lactide, or dimethyl cyclopolysiloxanes can be used.

These ring-opening copolymers from the ionically polymerizable cyclic compounds may be random or blocked copolymers.

As products available on the market of the polyether diols, there are, for example, PTMG650, PTMG1000 and PTMG2000 (each produced by Mitsubishi Kagaku Co., Ltd.); PPG700, PPG1000 and EXCENOL 2020 or 1020 (each produced by Asahi Glass Urethane Co., Ltd.); PEG1000, UNISAFEDC1100 and DC1800 (each produced by Nippon Yushi Co., Ltd.); PTG650, PTG1000, PTG2000, PTG3000, PPTG2000, PPTG1000, PTGL1000 and PTGL2000 (each produced by Hodogaya Kagaku Kogyo Co., Ltd.); and Z-3001-4, Z-3001-5, PBG2000 and PBG2000B (each produced by Daiichi Kogyo Seiyaku Co., Ltd.).

As a polyether polyol having in the molecule an alkyleneoxy structure, a diol represented by formula (DO1) is suitably used.

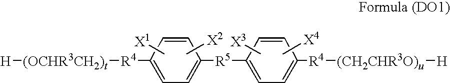

In formula above, R³ independently represents a hydrogen atom or a methyl group; R⁴ independently represents an oxygen atom or a sulfur atom; R⁵ represents —CH₂—, —C(CH₃)₂—, —S—, —SO— or —SO₂—; X¹ through X⁴ independently represent a hydrogen atom, a methyl group or a bromine atom; and t and u independently represent an integer of from 0 to 9′

In formula (DO1) above, it is preferred that t and u independently represent an integer of from 1 to 9.

Examples of diol represented by formula (DO1) include an adduct of bisphenol A with ethylene oxide (t=u=1.3), an adduct of bisphenol A with ethylene oxide (t=u=2), an adduct of bisphenol A with ethylene oxide (t=u=5), an adduct of bisphenol A with propylene oxide (t=u=1.1), an adduct of bisphenol A with propylene oxide (t=u=1.5), an adduct of bisphenol A with propylene oxide (t=u=3), an adduct of bisphenol F with ethylene oxide (t=u=2), an adduct of bisphenol F with ethylene oxide (t=u=4), an adduct of bisphenol F with propylene oxide (t=u=2), an adduct of bisphenol S with ethylene oxide (t=u=2), an adduct of bisphenol S with propylene oxide (t=u=2), and an adduct of tetrabromobisphenol A with ethylene oxide (t=u=2).

The preferred number average molecular weight of the diol represented by formula (DO1) is 1,000 or less in terms of polystyrene.

As products available on the market of the diol represented by formula (DO1), there are, for example, DA-400, DA-550, DA-700, DB-400, DB-530, DB-900, and DAB-800 (each produced by Nippon Yushi Co., Ltd.).

These polyether polyols may be used singly or as an admixture of two or more kinds thereof.

Besides the compounds described above, the following reaction products are also preferred as a polyether polyol having in the molecule an alkyleneoxy structure. M-14: A reaction product of 2-(2-hydroxyethyl)-piperidine (1 mole), hexane-1,6-diisocyanate (2 moles), 2-hydroxyethyl methacrylate (2 moles) and diethylene glycol (2 moles) M-15: A reaction product of 2-(2-hydroxyethyl)-piperidine (1 mole), tolylene-2,4-diisocyanate (2 moles), 2-hydroxyethyl methacrylate (2 moles) and diethylene glycol (2 moles) M-16: A reaction product of 2-(2-hydroxyethyl)-piperidine (i mole), tolylene-2,4-diisocyanate (2 moles), 2-hydroxyethyl methacrylate (2 moles) and tetraethylene glycol (2 moles)

M-17: A reaction product of 2-(2-hydroxyethyl)-piperidine (1 mole), tolylene-2,4-diisocyanate (2 moles), 2-hydroxyethyl methacrylate (2 moles) and 1,5-pentanediol (2 moles)
M-18: A reaction product of 2-(2-hydroxyethyl)-piperidine (1 mole), tolylene-2,4-diisocyanate (2 moles), 2-hydroxyethyl methacrylate (2 moles) and 1,4-cyclohexanedimethanol (2 moles)
M-19: A reaction product of N-n-butyldiethanolamine (1 mole), tolylene-2,4-diisocyanate (2 moles), 2-hydroxyethyl methacrylate (2 moles) and 1,4-cyclohexanedimethanol (2 moles)
M-20: A reaction product of N-n-butyldiethanolamine (1 mole), tolylene-2,4-diisocyanate (2 moles), 2-hydroxyethyl methacrylate (2 moles) and an adduct represented by formula below (R³═H, R⁴=O, R⁵=—C(CH₃)₂—, X¹ through X⁴═H, and t=u=2) of bisphenol A with ethylene oxide (2 moles)

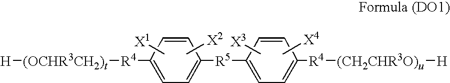

M-21: A reaction product of N-n-butyldiethanolamine (1 mole), tolylene-2,4-diisocyanate (2 moles), 2-hydroxyethyl methacrylate (2 moles) and an adduct represented by formula above (R³═H, R⁴═O, R⁵=—C(CH₃)₂—, X¹ through X⁴═H, and t=u=5) of bisphenol A with ethylene oxide (2 moles)
M-22: A reaction product of N-n-butyldiethanolamine (1 mole), tolylene-2,4-diisocyanate (2 moles), 2-hydroxyethyl methacrylate (2 moles) and an adduct represented by formula above (R³═H, R⁴═O, R⁵=—C(CH₃)₂—, X¹ through X⁴═H, and t=u=9) of bisphenol A with ethylene oxide (2 moles)

In the invention, various ethylenically unsaturated compounds other than the compounds described above can be used in combination. Such compounds are not specifically limited, but preferred examples thereof include a monofunctional acrylate such as 2-ethylhexyl acrylate, 2-hydroxypropyl acrylate, glycerol acrylate, tetrahydrofurfuryl acrylate, phenoxyethyl acrylate, nonylphenoxyethyl acrylate, tetrahydrofurfuryloxyethyl acrylate, tetrahydrofurfuryloxyhexyl acrylate, or 1,3-dioxolanyl acrylate; a methacrylate, itaconate, crotonate or maleate alternative of the above acrylate; a bifunctional acrylate such as ethyleneglycol diacrylate, triethyleneglycol diacrylate, pentaerythritol diacrylate, hydroquinone diacrylate, resorcin diacrylate, hexanediol diacrylate, neopentyl glycol diacrylate, tripropylene glycol diacrylate, hydroxypivalic acid neopentyl glycol diacrylate, neopentyl glycol adipate diacrylate, diacrylate of hydroxypivalic acid neopentyl glycol-ε-caprolactone adduct, 2-(2-hydroxy-1,1-dimethylethyl)-5-hydroxymethyl-5-ethyl-1,3-dioxane diacrylate, tricyclodecanedimethylol acrylate, tricyclodecanedimethylol acrylate-ε-caprolactone adduct or 1,6-hexanediol diglycidylether diacrylate; a dimethacrylate, diitaconate, dicrotonate or dimaleate alternative of the above diacrylate; a polyfunctional acrylate such as trimethylolpropane triacrylate, ditrimethylolpropane tetraacrylate, trimethylolethane triacrylate, pentaerythritol triacrylate, pentaerythritol tetraacrylate, dipentaerythritol tetraacrylate, dipentaerythritol pentaacrylate, dipentaerythritol hexacrylate, dipentaerythritol hexacrylate-ε-caprolactone adduct, pyrrogallol triacrylate, propionic acid dipentaerythritol triacrylate, propionic acid dipentaerythritol tetraacrylate, hydroxypivalylaldehyde modified dimethylolpropane triacrylate or EO-modified products thereof; and a methacrylate, itaconate, crotonate or maleate alternative of the above polyfunctional acrylate.

A prepolymer can be used as the ethylenically unsaturated compound.

Examples of the prepolymer include compounds described later and prepolymers with a photopolymerization property obtained by incorporating an acryloyl or methacryloyl group into a prepolymer with an appropriate molecular weight.

These prepolymers can be used singly or as an admixture of the above described monomers and/or oligomers.

Examples of the prepolymer include polyester (meth)acrylate obtained by incorporating (meth)acrylic acid in a polyester of a polybasic acid such as adipic acid, trimellitic acid, maleic acid, phthalic acid, terephthalic acid, hymic acid, malonic acid, succinic acid, glutaric acid, itaconic acid, pyromellitic acid, fumalic acid, pimelic acid, sebatic acid, dodecanic acid or tetrahydrophthalic acid with a polyol such as ethylene glycol, ethylene glycol, diethylene glycol, propylene oxide, 1,4-butane diol, triethylene glycol, tetraethylene glycol, polyethylene glycol, grycerin, trimethylol propane, pentaerythritol, sorbitol, 1,6-hexanediol or 1,2,6-hexanetriol; an epoxyacrylate such as bisphenol A-epichlorhydrin•(meth)acrylic acid or phenol novolak-epichlorhydrin•(meth)acrylic acid obtained by incorporating (meth)acrylic acid in an epoxy resin; an urethaneacrylate such as ethylene glycol-adipic acid tolylenediisocyanate•2-hydroxyethylacrylate, polyethylene glycol•tolylenediisocyanate-2-hydroxyethylacrylate, hydroxyethylphthalyl methacrylate-xylenediisocyanate, 1,2-polybutadieneglycol•tolylenediisocyanate•2-hydroxyethylacrylate or trimethylolpropane-propylene glycol-tolylenediisocyanate•2-hydroxyethylacrylate, obtained by incorporating (meth)acrylic acid in an urethane resin; a silicone acrylate such as polysiloxane acrylate, or polysiloxane-diisocyanate•2-hydroxyethylacrylate; an alkyd modified acrylate obtained by incorporating a methacroyl group in an oil modified alkyd resin; and a spiran resin acrylate.

Further, examples of the ethylenically unsaturated compounds include a monomer such as a phosphazene monomer, triethylene glycol, an EO modified isocyanuric acid diacrylate, an EO modified isocyanuric acid triacrylate, dimethyloltricyclodecane diacrylate, trimethylolpropane acrylate benzoate, an alkylene glycol acrylate or a urethane modified acrylate; and an addition polymerizable oligomer or prepolymer having a structural unit derived from the above monomer.

Besides the above compounds, compounds disclosed in Japanese Patent O.P.I. Publication Nos. 58-212994, 61-6649, 62-46688, 62-48589, 62-173295, 62-187092, 63-67189, and 1-244891, compounds described on pages 286 to 294 of “11290 Chemical Compounds” edited by Kagakukogyo Nipposha, and compounds described on pages 11 to 65 of “UV-EB Koka Handbook (Materials)” edited by Kobunshi Kankokai can be suitably used.

The ethylenically unsaturated compound content of the light sensitive layer is preferably from 1.0 to 80.0% by weight, and more preferably from 3.0 to 70.0% by weight.

### (Photopolymerization Initiator)

The light sensitive layer in the invention contains a photopolymerization initiator.

The photopolymerization initiator in the invention is a compound which initiates polymerization of an ethylenically unsaturated compound on light exposure. As the photopolymerization initiator is used a biimidazole compound, an iron arene complex, a titanocene compound, a polyhalogen compound or a monoalkyltriaryl borate compound. Among these, a biimidazole compound, an iron arene complex or a polyhalogen compound is preferred. In the invention, when polymerization initiated by infrared ray, at least a polyhalogen compound is used as a photopolymerization initiator. Other photopolymerization initiators can be also used besides theses photopolymerization initiator.

### [Polyhalogen Compound]

The polyhalogen compound in the invention is a compound having a trihalomethyl group, a dihalomethyl group or a dihalomethylene group. In the invention, an oxadiazole compound having in the molecule the group described above as the substituent or a polyhalogen compound represented by the following formula (1) is preferably used. A polyhalogen compound represented by the following formula (2) is more preferably used.

R¹—CY₂—(C═O)—R² Formula (1)

wherein R¹ represents a hydrogen atom, a halogen atom, an alkyl group, an aryl group, an acyl group, an alkylsulfonyl group, an arylsulfonyl group, an iminosulfo group or a cyano group; R² represents a monovalent substituent, provided that R¹ and R² may combine with each other to form a ring; and Y represents a halogen atom.

CY₃—(C═O)—X—R³ Formula (2)

wherein R³ represents a monovalent substituent; X represents —O— or —NR⁴— in which R⁴ represents a hydrogen atom or an alkyl group, provided that R³ and R⁴ may combine with each other to form a ring; and Y represents a halogen atom.

Among these, a polyhalogen compound having a polyhaloacetylamido group is preferably used.

Examples of a polyhalogen compound represented by formula (1) include compounds BR1 through BR 76 as shown later. An oxadiazole compound having in the molecule a polyhalomethyl group as a substituent is used, and examples thereof include H1 through H14 as shown later. Oxadiazole compounds disclosed in Japanese Patent O.P.I. Publication Nos. 5-34904, 5-45875 and 8-240909 is preferably used also.

In the invention, compounds in which a bromine atom in the above compounds is replaced with a chlorine atom are preferably used. Preferred examples of the polyhalogen compound in the invention will be listed below.

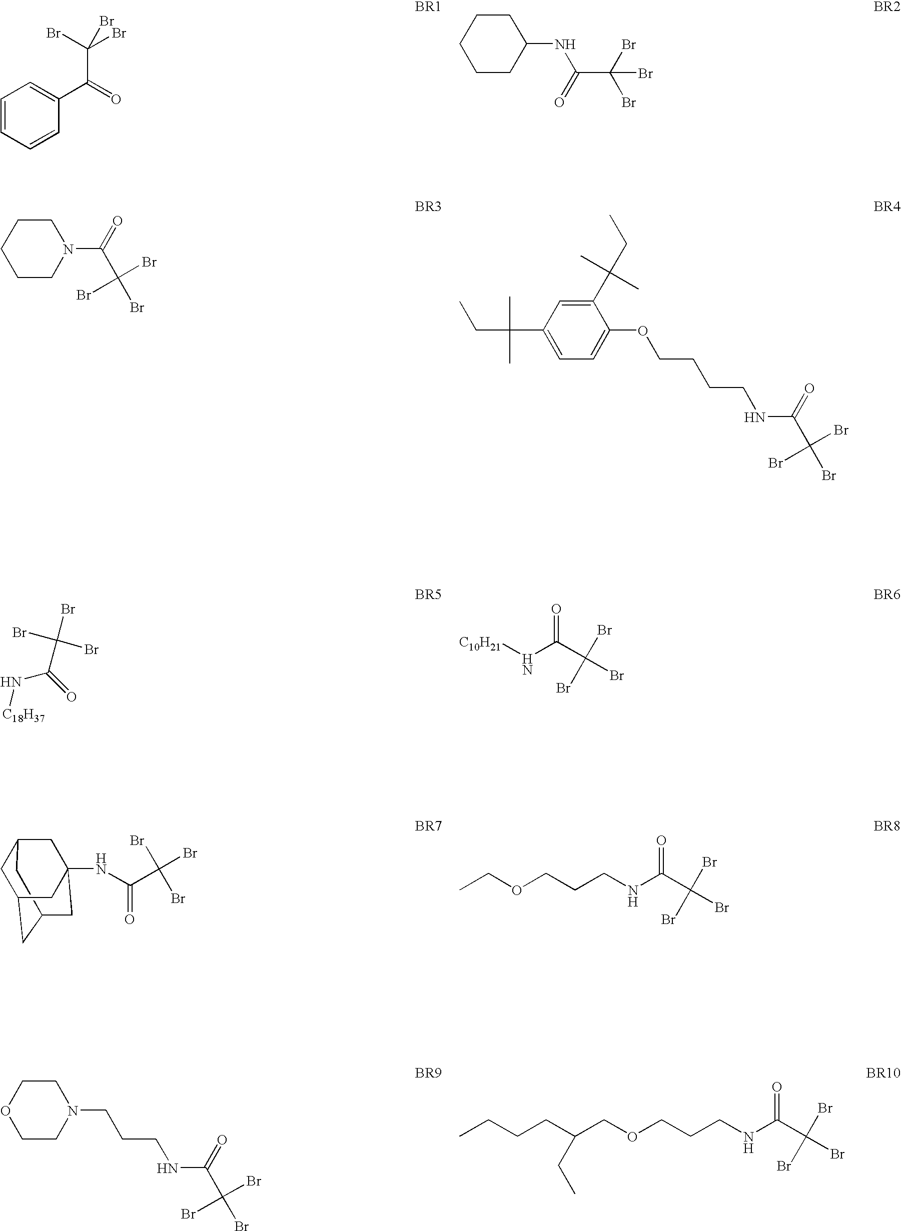

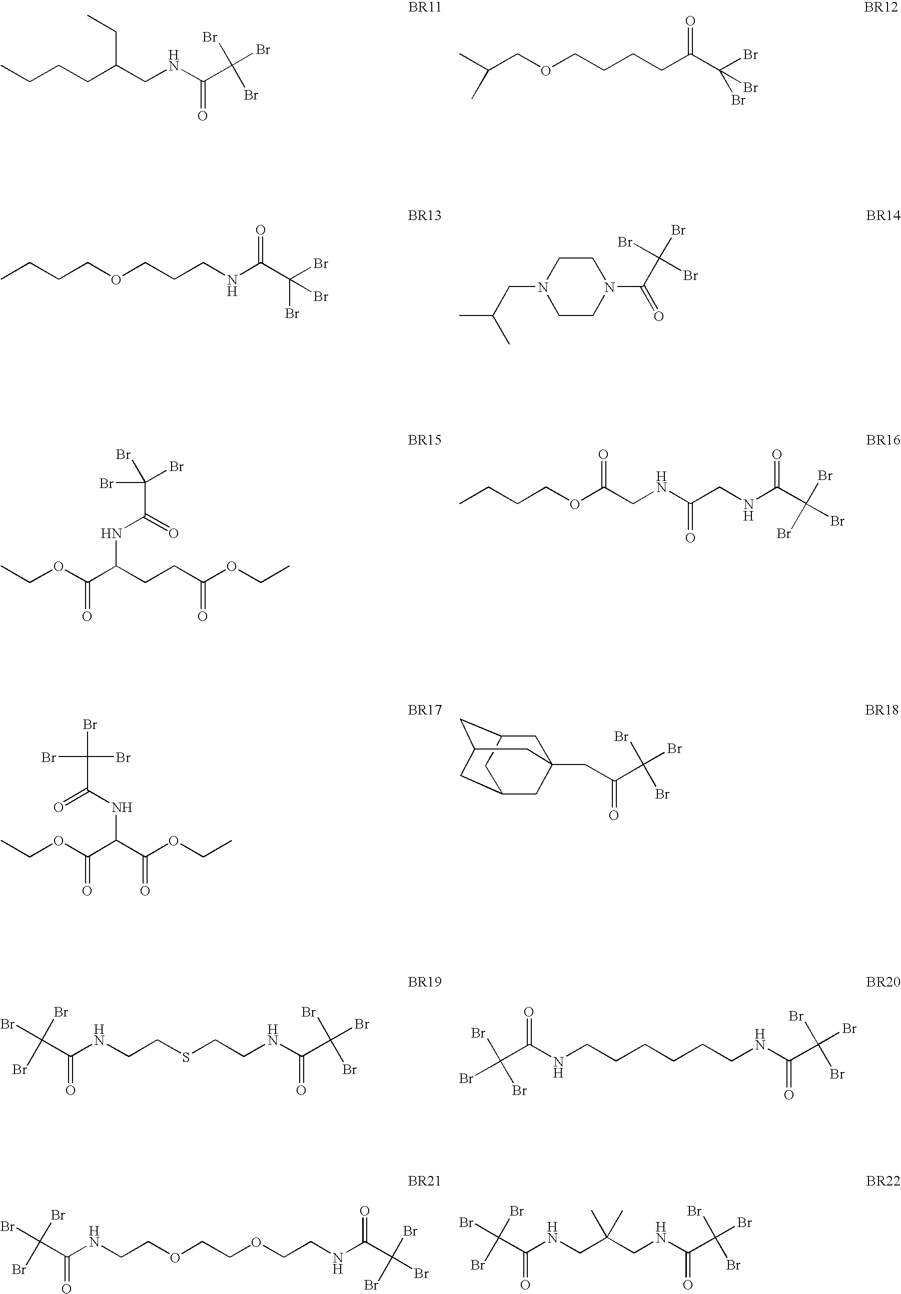

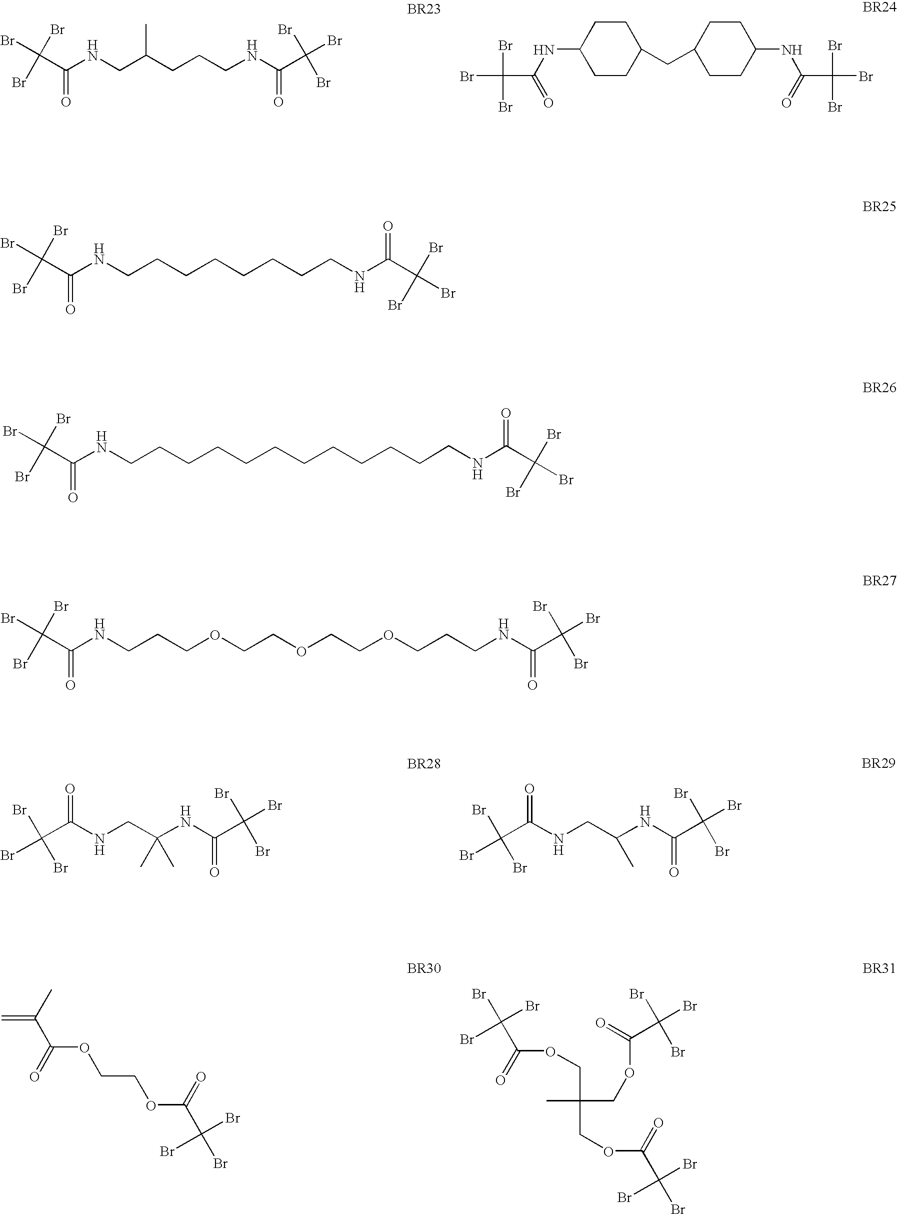

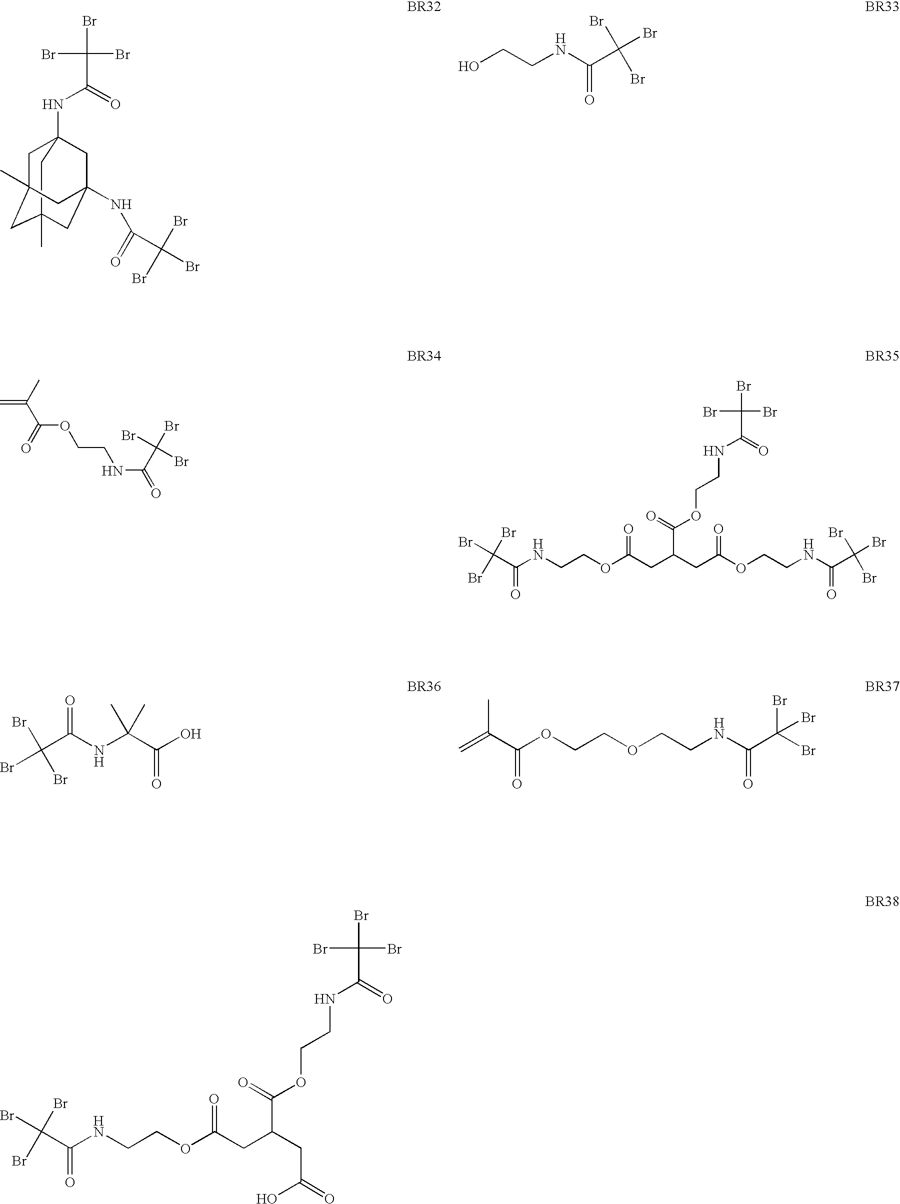

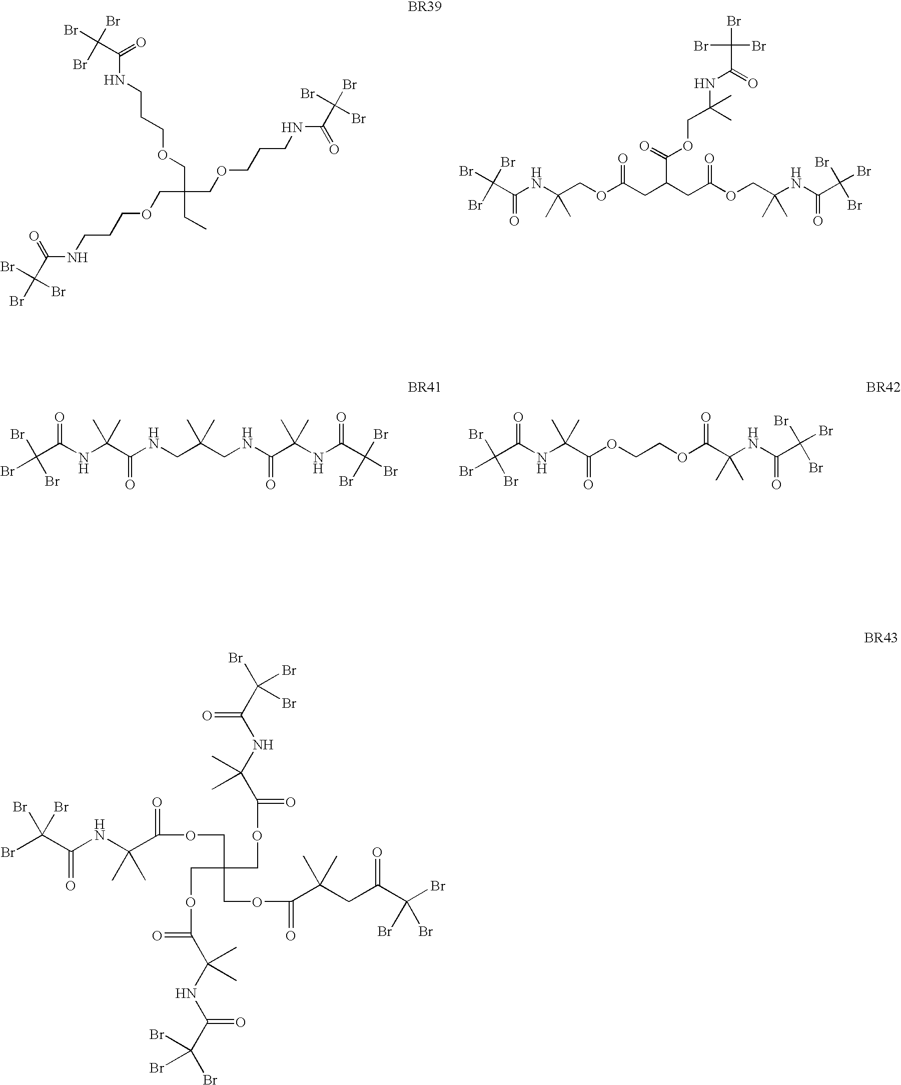

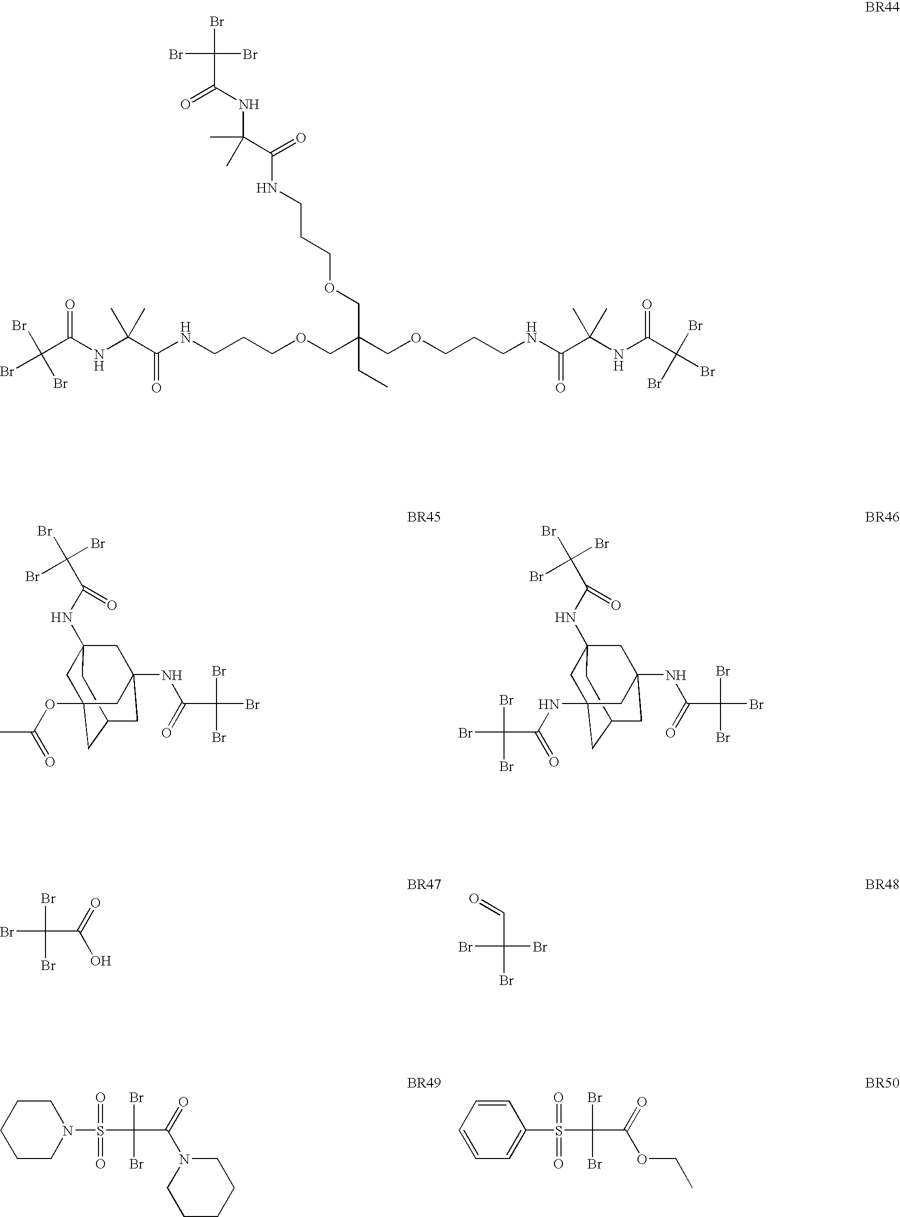

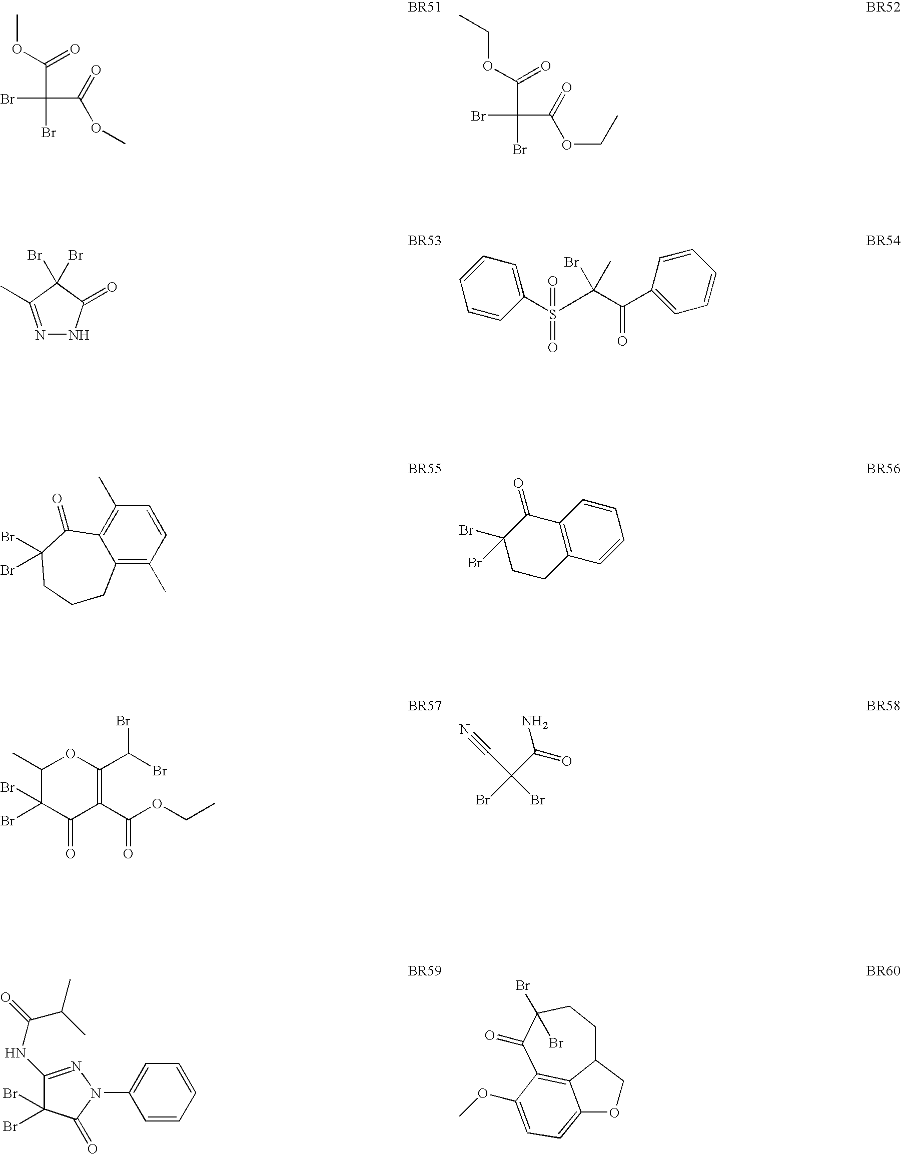

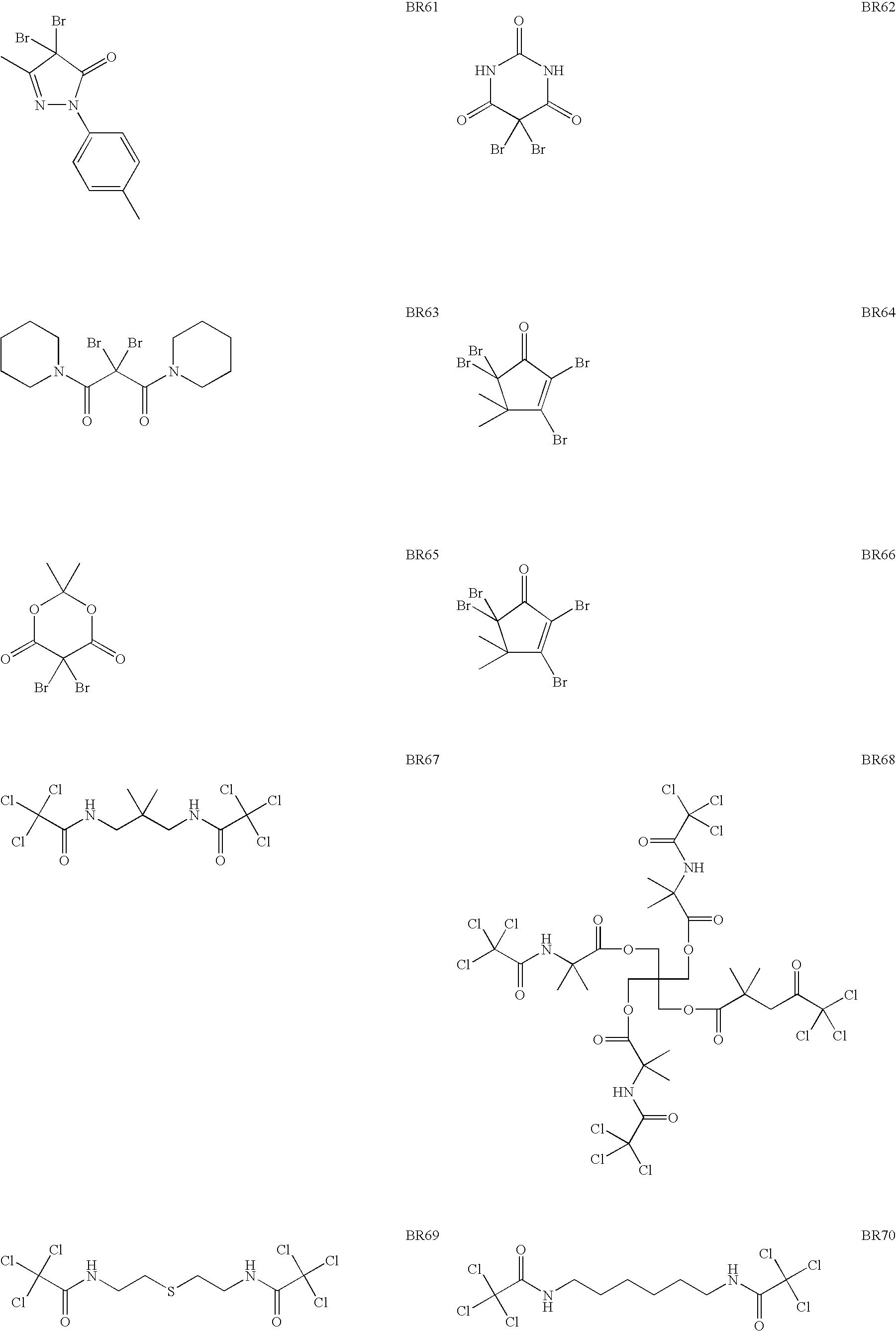

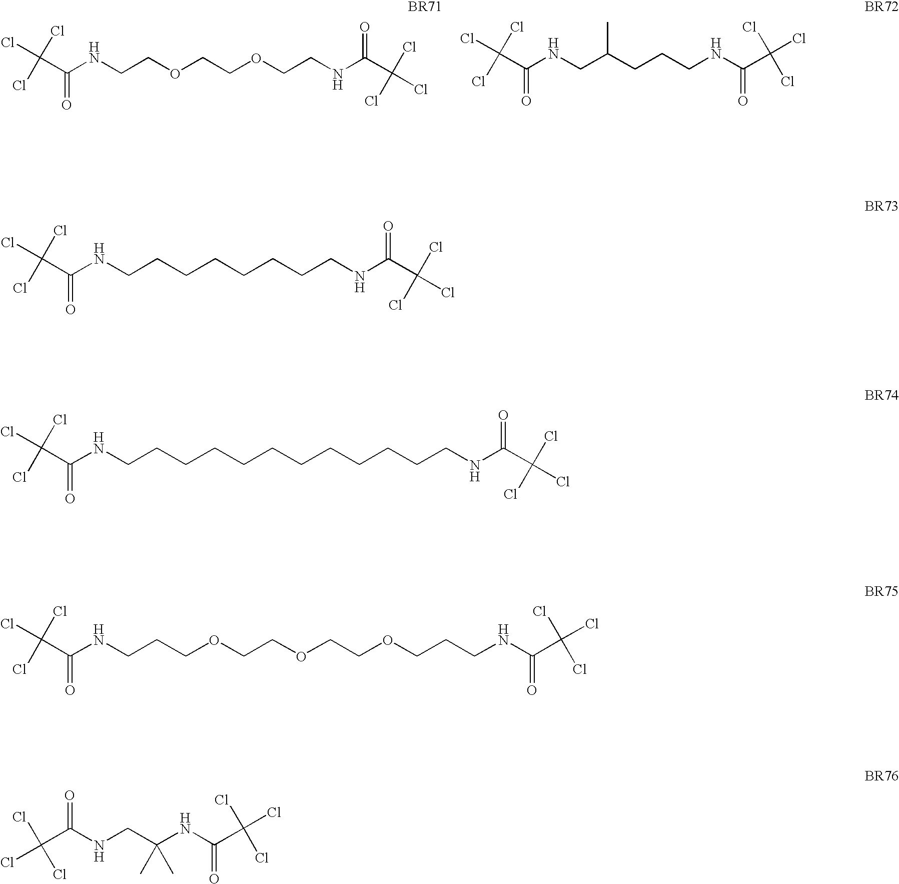

Formula (1)

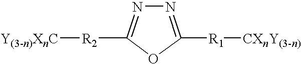

| Compound | R₁ | R₂ | X | n |
|---|---|---|---|---|
| H-1 | Single bond | Single bond | Cl | 3 |
| H-2 | Single bond | Single bond | Br | 3 |
| H-3 | CH₂ | CH₂ | Cl | 3 |
| H-4 | CH₂ | CH₂ | Br | 3 |
| H-5 | OCH₂ | OCH₂ | Cl | 3 |
| H-6 | OCH₂ | OCH₂ | Br | 3 |
| H-7 | O | O | Cl | 3 |
| H-8 | O | O | Br | 3 |
| H-9 | OCO | COO | Cl | 3 |
| H-10 | CH₂—COO | COO—CH₂ | Br | 3 |
| H-11 | NH—CO | CO—NH | Cl | 3 |
| H-12 | NH—CO | CO—NH | Br | 3 |
| H-13 | SO₂—NH | NH—SO₂ | Cl | 3 |
| H-14 | SO₂—NH | NH—SO₂ | Br | 3 |

The light sensitive layer in the invention may contain the following polymerization initiator in addition to the polyhalogen compound described above.

### [Biimidazole Compound]

The biimidazole compound is a derivative of biimidazole, and examples thereof include those disclosed in for example, Japanese Patent O.P.I. Publication No. 2003-295426.

In the invention, a hexaarylbisimidazole (HABI, a dimer of a triarylimidazole) compound is preferred as the biimidazole compound.

The synthetic method of the hexaarylbisimidazoles (HABI, dimmers of triarylimidazoles) is disclosed in DE 1470154, and use thereof in a photopolymerizable composition is disclosed in EP 24629, EP 107792, U.S. Pat. No. 4,410,621, EP 215453 and DE 3,211,312.

Preferred examples of the biimidazole compound include 2,4,5,2′,4′,5′-hexaphenylbisimidazole, 2,2′-bis(2-chlorophenyl)-4,5,4′,5′-tetraphenylbisimidazole, 2,2′-bis(2-bromophenyl)-4,5,4′,5′-tetraphenylbisimidazole, 2,2′-bis(2,4-dichlorophenyl)-4,5,4′,5′-tetraphenylbisimidazole, 2,2′-bis(2-chlorophenyl)-4,5,4′,5′-tetrakis(3-methoxyphenyl)bisimidazole, 2,2′-bis(2-chlorophenyl)-4,5,4′,5′-tetrakis(3,4,5-trimethoxyphenyl)bisimidazole, 2,5,2′,5′-tetrakis(2-chlorophenyl)-4,4′-bis(3,4-dimethoxyphenyl)bisimidazole, 2,2′-bis(2,6-dichlorophenyl)-4,5,4′,5′-tetraphenylbisimidazole, 2,2′-bis(2-nitrophenyl)-4,5,4′,5′-tetraphenylbisimidazole, 2,2′-di-o-tolyl-4,5,4′,5′-tetraphenylbisimidazole, 2,2′-bis(2-ethoxyphenyl)-4,5,4′,5′-tetraphenylbisimidazole, and 2,2′-bis(2,6-difluorophenyl)-4,5,4′,5′-tetraphenylbisimidazole.

The content of the HABI in a light sensitive composition is preferably from 0.01 to 30% by weight, and more preferably from 0.5 to 20% by weight, based on the nonvolatile components in the light sensitive composition.

As preferred photopolymerizable initiator used in combination, there are a titanocene compound, a monoalkyltriaryl borate compound and an iron arene complex.

As the titanocene compounds, there are those described in Japanese Patent O.P.I. Publication Nos. 63-41483 and 2-291. Preferred examples of the titanocene compounds include bis(cyclopentadienyl)-Ti-di-chloride, bis(cyclopentadienyl)-Ti-bis-phenyl, bis(cyclopentadienyl)-Ti-bis-2,3,4,5,6-pentafluorophenyl, bis(cyclopentadienyl)-Ti-bis-2,3,5,6-tetrafluorophenyl, bis(cyclopentadienyl)-Ti-bis-2,4,6-trifluorophenyl, bis(cyclopentadienyl)-Ti-bis-2,6-difluorophenyl, bis(cyclopentadienyl)-Ti-bis-2,4-difluorophenyl, bis(methylcyclopentadienyl)-Ti-bis-2,3,4,5,6-pentafluorophenyl, bis(methylcyclopentadienyl)-Ti-bis-2,3,5,6-tetrafluorophenyl, bis(methylcyclopentadienyl)-Ti-bis-2,6-difluorophenyl (IRUGACURE 784, produced by Ciba Speciality Chemicals Co.), bis(cyclopentadienyl)-bis(2,4,6-trifluoro-3-(pyry-1-yl)phenyl)titanium, and bis(cyclopentadienyl)-bis(2,4,6-trifluoro-3-(2-5-dimethylpyry-1-yl)phenyl)titanium.

As the monoalkyltriaryl borate compounds, there are those described in Japanese Patent O.P.I. Publication Nos. 62-150242 and 62-143044. Preferred examples of the monoalkyl-triaryl borate compounds include tetra-n-butyl ammonium n-butyltrinaphthalene-1-yl-borate, tetra-n-butylammonium n-butyltriphenylborate, tetra-n-butylammonium triphenyl-mono-t-butylborate, tetra-n-butylammonium n-butyl-tri-(4-tert-butylphenyl)borate, tetra-n-butylammonium n-hexyl-tri-(3-chloro-4-methylphenyl)borate, and tetra-n-butylammonium n-hexyl-tri-(3-fluorophenyl)borate.

As the iron arene complexes, there are those described in Japanese Patent O.P.I. Publication No. 59-219307.

Preferred iron arene complex is a compound represented by formula (a) below.

[A-Fe—B]+X⁻ Formula (a)

wherein A represents a substituted or unsubstituted cyclopentadienyl group or a substituted or unsubstituted cyclohexadienyl group; B represents a compound having an aromatic ring; and X⁻ is an anion.

Examples of the compound having an aromatic ring include benzene, toluene, xylene, cumene, naphthalene, 1-methylnaphtalene, 2-methylnaphtalene, biphenyl, fluorene, anthracene and pyrene. Examples of X— include PF₆⁻, BF₄⁻, SbF₆⁻, AlF₄⁻, and CF₃SO₃⁻. The substituents of the substituted or unsubstituted cyclopentadienyl group or a substituted or unsubstituted cyclohexadienyl group include an alkyl group such as methyl, ethyl, etc., a cyano group, an acetyl group and a halogen atom.

The content of the iron arene complex in a light sensitive composition is preferably from 0.1 to 20% by weight, and more preferably from 0.1 to 10% by weight, based on compound having a polymerizable group.

Examples of the iron arene complex include:

Fe-1: (η6-benzene)(η5-cyclopentadienyl)iron (2) hexafluorophosphate;
Fe-2: (η6-toluene)(η5-cyclopentadienyl)iron (2) hexafluorophosphate;
Fe-3: (η6-cumene)(η5-cyclopentadienyl)iron (2) hexafluorophosphate;
Fe-4: (η6-benzene)(η5-cyclopentadienyl)iron (2) hexafluoroarsenate;
Fe-5: (η6-benzene) (η5-cyclopentadienyl) iron (2) tetrafluoroborate;
Fe-6: (η6-naphthalene)(η5-cyclopentadienyl)iron (2) hexafluorophosphate;
Fe-7: (η6-anthracene)(η5-cyclopentadienyl)iron (2) hexafluorophosphate;
Fe-8: (η6-pyrene)(η5-cyclopentadienyl)iron (2) hexafluorophosphate;
Fe-9: (η6-benzene)(η5-cyanocyclopentadienyl)iron (2) hexafluorophosphate;
Fe-10: (η6-toluene)(η5-acetylcyclopentadienyl)iron (2) hexafluorophosphate;
Fe-11: (η6-cumene)(η5-cyclopentadienyl)iron (2) tetrafluoroborate;
Fe-12: (η6-benzene) (η5-carboethoxycyclohexadienyl) iron (2) hexafluorophosphate;
Fe-13: (η16-benzene)(η5-1,3-dichlorocyclohexadienyl)iron (2) hexafluorophosphate;
Fe-14: (η6-cyanobenzene)(η5-cyclohexadienyl)iron (2) hexafluorophosphate;
Fe-15: (η6-acetophenone)(η5-cyclohexadienyl)iron (2) hexafluorophosphate;
Fe-16: (η6-methyl benzoate)(η5-cyclopentadienyl)iron (2) hexafluorophosphate;
Fe-17: (η6-benzene sulfonamide)(η5-cyclopentadienyl)iron (2) tetrafluoroborate;
Fe-18: (η16-benzamide)(η5-cyclopentadienyl)iron (2) hexafluorophosphate;
Fe-19: (η6-cyanobenzene)(η5-cyanocyclopentadienyl)iron (2) hexafluorophosphate;
Fe-20: (η6-chloronaphthalene)(η5-cyclopentadienyl)iron (2) hexafluorophosphate;
Fe-21: (η6-anthracene)(η5-cyanocyclopentadienyl)iron (2) hexafluorophosphate;
Fe-22: (η6-chlorobenzene)(η5-cyclopentadienyl)iron (2) hexafluorophosphate;
Fe-23: (η6-chlorobenzene)(η5-cyclopentadienyl)iron (2) tetrafluoroborate;
Fe-24: (η6-fluorene)(η5-cyclopentadienyl)iron (2) hexafluorophosphate; and
Fe-25: (η16-xylene)(η5-cyclopentadienyl)iron hexafluorophosphate;

These compounds can be synthesized according to a method described in Dokl. Akd. Nauk. SSSR 149 615 (1963).

Another photopolymerization initiator can be used in combination. Examples thereof include carbonyl compounds, organic sulfur compounds, peroxides, redox compounds, azo or diazo compounds, halides and photo-reducing dyes disclosed in J. Kosar, “Light Sensitive Systems”, Paragraph 5, and those disclosed in British Patent No. 1,459,563.

Typical examples of the photopolymerization initiator used in combination include the following compounds:

A benzoin derivative such as benzoin methyl ether, benzoin i-propyl ether, or α,α-dimethoxy-α-phenylacetophenone; a benzophenone derivative such as benzophenone, 2,4-dichlorobenzophenone, o-benzoyl methyl benzoate, or 4,4′-bis(dimethylamino) benzophenone; a thioxanthone derivative such as 2-chlorothioxanthone, 2-i-propylthioxanthone; an anthraquinone derivative such as 2-chloroanthraquinone or 2-methylanthraquinone; an acridone derivative such as N-methylacridone or N-butylacridone; α,α-diethoxyacetophenone; benzil; fluorenone; xanthone; an uranyl compound; a triazine derivative disclosed in Japanese Patent Publication Nos. 59-1281 and 61-9621 and Japanese Patent O.P.I. Publication No. 60-60104; an organic peroxide compound disclosed in Japanese Patent O.P.I. Publication Nos. 59-1504 and 61-243807; a diazonium compound in Japanese Patent Publication Nos. 43-23684, 44-6413, 44-6413, 47-1604 and U.S. Pat. No. 3,567,453; an organic azide compound disclosed in U.S. Pat. Nos. 2,848,328, 2,852,379 and 2,940,853; orthoquinondiazide compounds disclosed in Japanese Patent Publication Nos. 36-22062b, 37-13109, 38-18015 and 45-9610; various onium compounds disclosed in Japanese Patent Publication No. 55-39162, Japanese Patent O.P.I. Publication No. 59-14023 and “Macromolecules”, Volume 10, p. 1307 (1977); azo compounds disclosed in Japanese Patent Publication No. 59-142205; metal arene complexes disclosed in Japanese Patent O.P.I. Publication No. 1-54440, European Patent Nos. 109,851 and 126,712, and “Journal of Imaging Science”, Volume 30, p. 174 (1986); (oxo) sulfonium organoboron complexes disclosed in Japanese Patent Application Nos. 4-56831 and 4-89535; transition metal complexes containing a transition metal such as ruthenium disclosed in “Coordination Chemistry Review”, Volume 84, p. 85-277 (1988) and Japanese Patent O.P.I. Publication No. 2-182701; 2,4,5-triarylimidazol dimmer disclosed in Japanese Patent O.P.I. Publication No. 3-209477; carbon tetrabromide; organic halide compounds disclosed in Japanese Patent O.P.I. Publication No. 59-107344.

The content of the polymerization initiator in the light sensitive layer is preferably from 0.1 to 20% by weight, and more preferably from 0.5 to 15% by weight, based on the total content of polymerizable ethylenically unsaturated compound.

### (Polymer Binder)

The polymer binder in the invention is one capable of carrying components contained in a light sensitive composition or a light sensitive layer, and various polymer binders which have been used in the art can be used.

As the polymer binder in the invention, there are a polyacrylate resin, a polyvinylbutyral resin, a polyurethane resin, a polyamide resin, a polyester resin, an epoxy resin, a phenol resin, a polycarbonate resin, a polyvinyl butyral resin, a polyvinyl formal resin, a shellac resin, or another natural resin. These resins can be used as an admixture of two or more thereof.

The polymer in the invention is preferably a copolymer obtained by copolymerization of acryl monomers, more preferably a copolymer containing (i) a carboxyl group-containing monomer unit and (ii) an alkyl methacrylate or alkyl acrylate unit, and still more preferably a copolymer containing a (meth)acrylic acid monomer unit in the molecule.

Examples of the carboxyl group-containing monomer include an α,β-unsaturated carboxylic acid, for example, acrylic acid, methacrylic acid, maleic acid, maleic anhydride, itaconic acid, itaconic anhydride or a carboxylic acid such as a half ester of phthalic acid with 2-hydroxymethacrylic acid.

Examples of the alkyl methacrylate or alkyl acrylate include an unsubstituted alkyl ester such as methyl methacrylate, ethyl methacrylate, propyl methacrylate, butyl methacrylate, amyl methacrylate, hexyl methacrylate, heptyl methacrylate, octyl methacrylate, nonyl methacrylate, decyl methacrylate, undecyl methacrylate, dodecyl methacrylate, methyl acrylate, ethyl acrylate, propyl acrylate, butyl acrylate, amyl acrylate, hexyl acrylate, heptyl acrylate, octyl acrylate, nonyl acrylate, decyl acrylate, undecyl acrylate, or dodecyl acrylate; a cyclic alkyl ester such as cyclohexyl methacrylate or cyclohexyl acrylate; and a substituted alkyl ester such as benzyl methacrylate, 2-chloroethyl methacrylate, N,N-dimethylaminoethyl methacrylate, glycidyl methacrylate, benzyl acrylate, 2-chloroethyl acrylate, N,N-dimethylaminoethyl acrylate or glycidyl acrylate.

The polymer in the invention can further contain, as another comonomer unit, a monomer unit derived from the monomer described in the following items 1) through 14):

1) A monomer having an aromatic hydroxy group, for example, o-, (p- or m-) hydroxystyrene, or o-, (p- or m-) hydroxyphenylacrylate;

2) A monomer having an aliphatic hydroxy group, for example, 2-hydroxyethyl acrylate, 2-hydroxyethyl methacrylate, N-methylolacrylamide, N-methylolmethacrylamide, 4-hydroxybutyl acrylate, 4-hydroxybutyl methacrylate, 5-hydroxypentyl acrylate, 5-hydroxypentyl methacrylate, 6-hydroxyhexyl acrylate, 6-hydroxyhexyl methacrylate, N-(2-hydroxyethyl)acrylamide, N-(2-hydroxyethyl)methacrylamide, or hydroxyethyl vinyl ether;

3) A monomer having an aminosulfonyl group, for example, m- or p-aminosulfonylphenyl methacrylate, m- or p-aminosulfonylphenyl acrylate, N-(p-aminosulfonylphenyl)methacrylamide, or N-(p-aminosulfonylphenyl)acrylamide;

4) A monomer having a sulfonamido group, for example, N-(p-toluenesulfonyl)acrylamide, or N-(p-toluenesulfonyl)-methacrylamide;

5) An acrylamide or methacrylamide, for example, acrylamide, methacrylamide, N-ethylacrylamide, N-hexylacrylamide, N-cyclohexylacrylamide, N-phenylacrylamide, N-nitrophenylacrylamide, N-ethyl-N-phenylacrylamide, N-4-hydroxyphenylacrylamide, or N-4-hydroxyphenylmethacrylamide;

6) A monomer having a fluorinated alkyl group, for example, trifluoromethyl acrylate, trifluoromethyl methacrylate, tetrafluoropropyl methacrylate, hexafluoropropyl methacrylate, octafluoropentyl acrylate, octafluoropentyl methacrylate, heptadecafluorodecyl methacrylate, heptadecafluorodecyl methacrylate, or N-butyl-N-(2-acryloxyethyl)heptadecafluorooctylsulfonamide;

7) A vinyl ether, for example, ethyl vinyl ether, 2-chloroethyl vinyl ether, propyl vinyl ether, butyl vinyl ether, octyl vinyl ether, or phenyl vinyl ether;

8) A vinyl ester, for example, vinyl acetate, vinyl chroloacetate, vinyl butyrate, or vinyl benzoate;

9) A styrene, for example, styrene, methylstyrene, or chloromethystyrene;

10) A vinyl ketone, for example, methyl vinyl ketone, ethyl vinyl ketone, propyl vinyl ketone, or phenyl vinyl ketone;

11) An olefin, for example, ethylene, propylene, isobutylene, butadiene, or isoprene;

12) N-vinylpyrrolidone, N-vinylcarbazole, or N-vinylpyridine,

13) A monomer having a cyano group, for example, acrylonitrile, methacrylonitrile, 2-pentenenitrile, 2-methyl-3-butene nitrile, 2-cyanoethyl acrylate, or o-, m- or p-cyanostyrene;

14) A monomer having an amino group, for example, N,N-diethylaminoethyl methacrylate, N,N-dimethylaminoethyl acrylate, N,N-dimethylaminoethyl methacrylate, polybutadiene urethane acrylate, N,N-dimethylaminopropyl acrylamide, N,N-dimethylacrylamide, acryloylmorpholine, N-isopropylacrylamide, or N,N-diethylacrylamide.

Further still another monomer may be employed as a comonomer.

The polymer binder in the invention is preferably a polymer having, in the side chain, a carboxyl group and a polymerizable double bond. As the polymer binder is also preferred an unsaturated bond-containing copolymer which is obtained by reacting a copolymer having a carboxyl group in the molecule with for example, a compound having a (meth)acryloyl group and an epoxy group.

Examples of the compound having a (meth)acryloyl group and an epoxy group in the molecule include glycidyl acrylate, glycidyl methacrylate and an epoxy group-containing unsaturated compound disclosed in Japanese Patent O.P.I. Publication No. 11-271969. Further, an unsaturated bond-containing copolymer is obtained by reacting a copolymer molecule having a hydroxyl group in the molecule with for example, a compound having a (meth)acryloyl group and an isocyanate group. Examples of the compound having a (meth)acryloyl group and an isocyanate group in the molecule include vinyl isocyanate, (meth)acryl isocyanate, 2-(meth)acroyloxyethyl isocyanate, m- or p-isopropenyl-α,α′-dimethylbenzyl isocyanate. Among these, (meth)acryl isocyanate or 2-(meth)acroyloxyethyl isocyanate is preferred.

Reaction of a carboxyl group-containing copolymer with a compound having in the molecule a (meth)acryloyl group and an epoxy group can be carried out according to a well-known method. For example, the reaction is carried out at a temperature of 20 to 100° C., and preferably 40 to 80° C., and more preferably at a boiling point of solvent used (while refluxing), for 2 to 10 hours and preferably 3 to 6 hours. As the solvent used in the reaction, there are solvents used in the polymerization to obtain the copolymer described above. After polymerization, the solvent in the polymerization can be used without being removed from the polymerization solution as a reaction solvent used for reaction in which an aliphatic epoxy group-containing unsaturated compound is incorporated into the vinyl copolymer. The reaction can be carried out in the presence of a catalyst or a polymerization inhibitor.

As the catalyst, amines or ammonium chlorides are preferred. Examples of the amines include triethylamine, tributylamine, dimethylaminoethanol, diethylaminoethanol, methylamine, ethylamine, n-propylamine, i-propylamine, 3-methoxypropylamine, butylamine, allylamine, hexylamine, 2-ethylhexylamine, and benzylamine. Examples of the ammonium chlorides include triethylbenzylammonium chloride.

The amount used of the catalyst is ordinarily from 0.01 to 20.0% by weight based on the weight of an aliphatic epoxy group-containing unsaturated compound used. Examples of the polymerization inhibitor include hydroquinone, hydroquinone monomethyl ether, tert-butylhydroquinone, 2,5-di-t-butylhydroquinone, methyl hydroquinone, 2,6-di-t-butylhydroquinone, p-benzoquinone, methyl-p-benzoquinone, tert-butyl-p-benzoquinone, and 2,5-diphenyl p-benzoquinone. The amount used of the polymerization inhibitor is ordinarily from 0.01 to 5.0% by weight based on the weight of aliphatic epoxy group-containing unsaturated compound used. The reaction process is controlled by measurement of acid value of the reaction mixture and the reaction is terminated at the time when the acid value is zero.

Reaction of a hydroxyl group-containing copolymer with a compound having in the molecule a (meth)acryloyl group and an isocyanate group can be carried out according to a known method. For example, the reaction is carried out at a temperature of 20 to 100° C., and preferably 40 to 80° C., and more preferably at a boiling point of solvent used (while refluxing), for 2 to 10 hours and preferably 3 to 6 hours. As the solvent used in the reaction, there are solvents used in the polymerization to obtain the vinyl copolymer above. After polymerization, the solvent in the polymerization can be used without being removed from the polymerization solution as a reaction solvent used for reaction in which an isocyanate group-containing unsaturated compound is incorporated into the polymer. The reaction can be carried out in the presence of a catalyst or a polymerization inhibitor. As the catalyst, tin compounds or amines are preferably used. Examples of thereof include dibutyltin laurate, and triethylamine.

The amount used of the catalyst is preferably from 0.01 to 20.0% by weight based on the weight of a double bond-containing compound used. Examples of the polymerization inhibitor include hydroquinone, hydroquinone monomethyl ether, tert-butylhydroquinone, 2,5-di-t-butylhydroquinone, methyl hydroquinone, 2,6-di-t-butylhydroquinone, p-benzoquinone, methyl-p-benzoquinone, tert-butyl-p-benzoquinone, and 2,5-diphenyl p-benzoquinone. The amount used of the polymerization inhibitor is ordinarily from 0.01 to 5.0% by weight based on the weight of isocyanate group-containing unsaturated compound used. The reaction process is controlled by measurement of infrared absorption spectra (IR) of the reaction mixture and the reaction is terminated at the time when the isocyanate absorption disappears.

The content of the polymer having in the side chain a carboxyl group and a polymerizable double bond is preferably from 50 to 100% by weight, and more preferably 100% by weight, based on the total weight of the polymeric binder used.

The polymer binder content of the light sensitive layer is preferably from 10 to 90% by weight, more preferably from 15 to 70% by weight, and still more preferably from 20 to 50% by weight, in view of sensitivity.

### [Water-Soluble Polymer Binder]

In the invention, the water-soluble polymer described below is preferably used as the water-soluble polymer binder.

Particularly in infrared ray-induced polymerization, the water-soluble polymer binder described later is preferably used as the polymer binder. Use of such a water-soluble polymer binder is extremely effective when printing is carried out without development process (so-called on-press development is carried out), which is preferred embodiment of the invention.

The water-soluble polymer binder in the invention means a water-soluble polymer having a water solubility of at least 0.1 and a molecular weight (weight average molecular weight) of at least 500. Herein, the water solubility is specified by the saturation concentration (grams) of a solute soluble in 100 g of 25° C. water.

In the invention, various known water-soluble polymers can be employed. Examples thereof include PVA-maleic acid copolymer disclosed in Japanese Patent O.P.I. Publication No. 48-97602, PVA-acrylamide copolymer disclosed in Japanese Patent O.P.I. Publication No. 48-97603, PVA incorporated with a reactive group disclosed in Japanese Patent O.P.I. Publication No. 2000-181062, polymers having in the side chain a cationic group and a methacryloyl group disclosed in Japanese Patent O.P.I. Publication No. 9-101620, and cationic water-soluble resins or sulfonate salt-containing resins disclosed in Japanese Patent O.P.I. Publication No. 2003-215801.

Examples of the water-soluble polymer in the invention include polyvinyl alcohol having various degrees of saponification, a homopolymer or copolymer of hydroxystyrene, a polyamide resin, a homopolymer or copolymer of vinyl pyrrolidone, polyethylene oxide, polyethylene imine, polyacrylamide, corn starch, mannan, pectin, agar, dextran, pullulan, glue, hydroxymethyl-cellulose, alginic acid, carboxymethylcellulose, and sodium polyacrylate.

Specifically, a polymer having a nonionic hydrophilic group is preferably used as the water-soluble polymer in the invention.

The water-soluble polymer has a weight average molecular weight of preferably from 1,000 to 100,000, and more preferably from 1,000 to 50,000, in view of printing durability and image reproduction.

The light sensitive layer in the invention may contain a compound other than the water-soluble polymer in the invention as a binder, but the content of the water-soluble polymer in the invention is preferably from 80 to 100% by weight, and more preferably from 90 to 100% by weight, based on the total weight of the binder.

The binder content of the image formation layer in the invention is preferably from 10 to 95% by weight, and more preferably from 30 to 90% by weight, based on the weight of light sensitive layer.

Examples of the binder used in combination include a polyvinyl butyral resin, a polyester resin, an epoxy resin, a phenol resin, a polycarbonate resin, a polyvinyl formal resin, shellac and other natural resins.

### (Polymer Having Nonionic Hydrophilic Group)

The nonionic hydrophilic group contained in the polymer described above having a nonionic hydrophilic group means a group or bond exhibiting hydrophilicity without being ionized in water, and examples thereof include an alcoholic hydroxyl group, an aromatic hydroxyl group, an acid amide group, a sulfonamide group, a thiol group, a pyrrolidone group, a polyoxyethylene group, a polyoxypropylene group, a sugar residue and so forth.

As the polymer having a nonionic hydrophilic group, one containing a nonionic hydrophilic group of at least 300 by weight is preferred in view of developability.

The polymer containing the above-described nonionic hydrophilic group is preferably an oligomer or a polymer having a weight average molecular weight of from 1,000 to 50,000, in view of developability and image reproduction, and examples thereof include a polymer obtained by polymerizing at least one unsaturated monomer having the foregoing nonionic hydrophilic group in the side chain, a polyvinyl alcohol based polymer, and a cellulose based polymer as polysaccharide, and a glucose based polymer.

Examples of the unsaturated monomer having an amide group in the side chain include unsubstituted or substituted (meth)acrylamide; an amidated monomer of a dibasic acid such as an itaconic acid, a fumaric acid or a maleic acid; N-vinylacetoamide; N-vinylformamide; N-vinylpyrrolidone and so forth.

Typical examples of unsubstituted or substituted (meth)acrylamide include (meth)acrylamide, N-methyl(meth)acrylamide, N,N-dimethyl(meth)acrylamide, N-ethyl(meth)acrylamide, N,N-diethyl(meth)acrylamide, N,N-dimethylaminopropyl(meth)acrylamide, N-isopropyl(meth)acrylamide, diacetone(meth)acrylamide, methylol(meth)acrylamide, methoxymethyl(meth)acrylamide, butoxymethyl(meth)acrylamide, sulfonic acid propyl(meth)acrylamide and (meth)acryloylmorpholine.

The amidated monomer of a dibasic acid such as the foregoing itaconic acid and so forth may be a monomide in which one carboxyl group is amidated, a diamide in which both carboxyl groups are amidated, or an amideester in which one carboxyl group is amidated, and the other carboxyl group is esterified.

Examples of the unsaturated monomer having a hydroxyl group include hydroxyethyl(meth)acrylate, hydroxypropyl(meth)acrylate, hydroxybutyl(meth)acrylate, a monomer in which ethyleneoxide or propyleneoxide is added thereto, methylol(meth)acrylamide, and methoxymethyl(meth)acrylamide and butoxymethyl-(meth)acrylamide, which are a condensation product of the methylol(meth)acrylamide with methyl alcohol or butyl alcohol.

The foregoing “(meth)acryl”, “(meth)acrylate” and “(meth)acryloyl” mean acryl or methacryl, acrylate or methacrylate, and acryloyl or methacryloyl, respectively.

A polyvinyl alcohol based polymer will further be described in detail. Examples thereof include a polymer obtained via complete or partial hydrolysis of a homopolymer or a copolymer of a fatty acid vinyl monomer such as vinyl acetate or vinyl propionate, and a polymer obtained by partial formalization, acetalization or butyralization of the foregoing polymer.

The polymer containing a nonionic hydrophilic group of at least 30% by weight may possess a crosslinking functional group reacting with a crosslinking agent. The crosslinking functional group is preferably a nonionic one, depending on kinds of the crosslinking agent, and examples thereof include a hydroxyl group, an isocyanate group, a glycidyl group, an oxazolyl group and so forth. In order to introduce these crosslinking functional groups, an unsaturated monomer having the functional group such as a unsaturated monomer having the foregoing hydroxyl group or glycidyl(meth)acrylate as a unsaturated monomer having the glycidyl group may be copolymerized with another (meth)acrylate monomer.

A polymer containing a nonionic hydrophilic group of 30% by weight may be a compound obtained by copolymerization of an unsaturated monomer having the foregoing nonionic hydrophilic group and an unsaturated monomer having a crosslinking functional group with another unsaturated co-monomer in order to further produce the effect of the present invention.

Examples of another unsaturated comonomer include methyl(meth)acrylate, ethyl(meth)acrylate, butyl(meth)acrylate, 2-ethylhexyl(meth)acrylate, glycidyl(meth)acrylate, methoxy(C1-C50)ethyleneglycol (meth)acrylate, dimethylaminoethyl(meth)acrylate, diethylaminoethyl(meth)acrylate, phenoxyethyl(meth)acrylate, benzyl(meth)acrylate, isopronyl (meth)acrylate, adamantly (meth)acrylate, cyclohexyl (meth)acrylate, styrene, α-methylstyrene, acrylonitrile, methacrylonitrile, vinyl acetate and α-olefin (C4-C30).

As a crosslinking agent used for crosslinking the polymer containing a nonionic hydrophilic group of at least 30% by weight, a combination of the crosslinking agents and the functional group as used in reaction described in “Crosslinking agent Handbook” (edited by T. Kaneko and S. Yamashita and published in 1981 by Taisei Co., Ltd.) can be selected Examples of the crosslinking agent include polyhydric alcohols, polycarboxylic acids and their anhydrides, polyglycidyl compounds (epoxy resins), polyamine compounds, polyamide resins, polyisocyanate compounds (including blocked isocianates), oxazoline resins; amino resins, and glyoxals, which are capable of reacting with a hydroxyl group, a glycidyl group or optionally an amido group as a crosslinking functional group in a polymeric compound containing a nonionic hydrophilic group of at least 30% by weight.

Among the foregoing crosslinking agents, commonly known various glycidyl compounds (epoxy resins), a hardener for an epoxy resin such as oxazoline resins, amino resins, polyamine compounds, polyamide resins and glyoxals are preferred in view of developability and printing properties.

Examples of the amino resins include a commonly known melamine resin, a urea resin, a benzoguanamine resin, a glycoluryl resin and modified resins thereof such as a carboxy-modified melamine resin and so forth. Further, in order to accelerate crosslinking reaction, tertiary amines may be used in combination in the case of employing the foregoing glycidyl compound, and an acidic compound such as a p-toluenesulfonic acid, a dodecylbenzenesulfonic acid or an ammonium chloride may be used in combination in the case of employing an amino resin. When a light sensitive composition is heated through heated air, a heated roller or a laser, these crosslinking agents in the composition are reacted with a compound containing a nonionic hydrophilic group of at least 30% by weight to crosslink the compound.

Examples of the polymer containing at least 30% by weight of a nonionic hydrophilic group will be listed below.

1. Vinyl pyrrolidone-vinyl acetate copolymer (60/40) with a weight average molecular weight of 34000

Product name: Luviskol 64 produced by BASF Japan, VP/VA=60% by weight/40% by weight copolymer

2. Vinyl pyrrolidone-1-butene copolymer (90/10) weight with a weight average molecular weight of 17000

Product name: GANPX P-904 LC ISP Chemicals

3. Vinyl pyrrolidone-glycidyl methacrylate copolymer (70/30) with a weight average molecular weight of 10000

4. Polyacrylamide with a weight average molecular weight of 1700

Product name: Acoflock N104 produced by Mitsui Chemicals AquaPolymer, Inc.

### (Spectral Sensitizing Agent)

The planographic printing plate material of the invention preferably contains a spectral sensitizing agent.

The spectral sensitizing agent increases sensitivity of a polymerization initiator to imagewise exposure. Various spectral sensitizing agents, which are used in the field pertaining to a light sensitive planographic printing plate material, can be employed according to the wavelength region (ultraviolet, visible or infrared) of light used. For example, a spectral sensitizing agent is preferably employed which absorbs light of the same wavelength region as the emission wavelength of light from a high power semiconductor laser as a light source. In the invention, a spectral sensitizing agent having absorption maximum in the range from 300 to 1200 nm is preferably employed.

In the invention, a spectral sensitizing agent may be a compound having a function capable of transferring energy obtained by absorption of light in the form of light (electromagnetic wave) to a polymerization initiator or a compound having a function capable of converting the energy to heat and transferring the heat to a polymerization initiator. Further, a spectral sensitizing agent may have both of the above functions.

In the invention, laser can be used as a light source for recording. Examples of the laser will be described later. When recording is carried out employing a semiconductor laser called a violet laser emitting light with an emission wavelength of from 350 to 450 nm, a spectral sensitizing agent having absorption maximum in the range of from 350 to 450 nm is preferably used. The spectral sensitizing agent having absorption maximum in the range of from 350 to 450 nm has is not specifically limited to the chemical structure, as long as it has the absorption maximum as described above. Examples thereof include spectral sensitizing agents disclosed in Japanese Patent O.P.I. Publication Nos. 2002-296764, 2002-268239, 2002-268238, 2002-268204, 2002-221790, 2002-202598, 2001-42524, 2000-309724, 2000-258910, 2000-206690, 2000-147763, and 2000-98605, but are not limited thereto.

Examples of the spectral sensitizing agent having absorption maximum in the range of from 350 to 450 nm include cyanine, merocyanine, porphyrin, a spiro compound, ferrocene, fluorene, fulgide, imidazole, perylene, phenazine, phenothiazine, acridine, an azo compound, diphenylmethane, triphenylmethane, triphenylamine, cumarin derivatives, quinacridone, indigo, styryl, pyrylium compounds, pyrromethene compounds, pyrazolotriazole compounds benzothiazole compounds, barbituric acid derivatives, thiobarbituric acid derivatives, and ketoalcohol borate complexes.

Among these spectral sensitizing agents, a coumarin-type spectral sensitizing agent represented by formula (SS-1) below is preferably used.

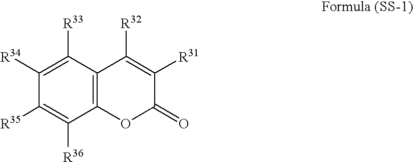

In formula above, R³¹ through R³⁶ independently represent a hydrogen atom or a substituent. Examples of the substituent include an alkyl group (for example, a methyl group, an ethyl group, a propyl group, an isopropyl group, a t-butyl group, a pentyl group, a hexyl group, an octyl group, a dodecyl group, a tridecyl group, a tetradecyl group, or a pentadecyl group), a cycloalkyl group (for example, a cyclopentyl group or a cyclohexyl group), an alkenyl group (for example, a vinyl group or a allyl group), an alkinyl group (for example, a propargyl group), an aryl group (for example, a phenyl group, or a naphthyl group), a heteroaryl group (for example, a furyl group, a thienyl group, a pyridazinyl group, a pyrimidinyl group, a pyrazinyl group, a triazinyl group, an imidazolyl group, a pyrazolyl group, a thiazolyl group, a benzimidazolyl group, a benzoxazolyl group, a quinazolyl group, or a phthalazinyl group), a saturated heterocyclic group (for example, a pyrrolidinyl group, an imidazolidinyl group, a morpholinyl group or an oxazolidinyl group), an alkoxy group (for example, a methoxy group, an ethoxy group, a propoxy group, a pentyloxy group, a hexyloxy group, an octyloxy group, or a dodecyloxy group), a cycloalkoxy group (for example, a cyclopentyloxy group, or a cyclohexyloxy group), an aryloxy group (for example, a phenoxy group or a naphthyloxy group), an alkylthio group (for example, a methylthio group, an ethylthio group, a propylthio group, a pentylthio group, a hexylthio group, an octylthio group, or a dodecylthio group), a cycloalkylthio group (for example, a cyclopentylthio group or a cyclohexylthio group), an arylthio group (for example, a phenylthio group, or a naphthylthio group), an alkoxycarbonyl group (for example, a methyloxycarbonyl group, an ethyloxycarbonyl group, a butyloxycarbonyl group, an octyloxycarbonyl group, or a dodecyloxycarbonyl group), an aryloxycarbonyl group (for example, a phenyloxycarbonyl group, or a naphthyloxycarbonyl group), a sulfamoyl group (for example, an aminosulfonyl group, a methylaminosulfonyl group, a dimethylaminosulfonyl group, a butylaminosulfonyl group, a hexylaminosulfonyl group, a cyclohexylaminosulfonyl group, an octylaminosulfonyl group, a dodecylaminosulfonyl group, a phenylaminosulfonyl group, a naphthylaminosulfonyl group, or a 2-pyridylaminosulfonyl group), an acyl group (for example, an acetyl group, an ethylcarbonyl group, a propylcarbonyl group, a pentylcarbonyl group, a cyclohexylcarbonyl group, an octylcarbonyl group, a 2-ethylhexylcarbonyl group, a dodecycarbonyl group, a phenylcarbonyl group, a naphthylcarbonyl group, or a pyridylcarbonyl group), an acyloxy group (for example, an acetyloxy group, an ethylcarbonyloxy group, a butylcarbonyloxy group, an octylcarbonyloxy group, a dodecycarbonyloxy group, or a phenylcarbonyloxy group), an amido group (for example, a methylcarbonylamino group, an ethylcarbonylamino group, a dimethylcarbonylamino group, a propylcarbonylamino group, a pentylcarbonylamino group, a cyclohexylcarbonylamino group, 2-ethylhexylcarbonylamino group, an octylcarbonylamino group, a a dodecycarbonylamino group, a phenylcarbonylamino group, a naphthylcarbonylamino group, or a pyridylcarbonyl group), a carbamoyl group (for example, an aminocarbonyl group, a methylaminocarbonyl group, a dimethylaminocarbonyl group, a propylaminocarbonyl group, a pentylaminocarbonyl group, a cyclohexylaminocarbonyl group, an octylaminocarbonyl group, a 2-ethylhexylaminocarbonyl group, a dodecyaminocarbonyl group, a phenylaminocarbonyl group, a naphthylaminocarbonyl group, or a 2-pyridylaminocarbonyl group), a ureido group (for example, a methylureido group, an ethylureido group, a pentylureido group, a cyclohexylureido group, an octylureido group, a dodecylureido group, a phenylureido group, a naphthylureido group, or a 2-pyridylureido group), a sulfinyl group (for example, a methylsulfinyl group, an ethylsulfinyl group, a butylsulfinyl group, a cyclohexylsulfinyl group, a 2-ethylhexylsulfinyl group, a dodecylsulfinyl group, a phenylsulfinyl group, a naphthylsulfinyl group, or a 2-pyridylsulfinyl group), an alkylsulfonyl group (for example, a methylsulfonyl group, an ethylsulfonyl group, a butylsulfonyl group, a cyclohexylsulfonyl group, a 2-ethylhexylsulfonyl group, or a dodecylsulfonyl group), an arylsulfonyl group (for example, a phenylsulfonyl group, a naphthylsulfonyl group, or a 2-pyridylsulfonyl group), an amino group (for example, an amino group, an ethylamino group, a dimethylamino group, a butylamino group, a cyclopentylamino group, a 2-ethylhexylamino group, a dodecylamino group, an anilino group, a naphthylamino group, or a 2-pyridylamino group), a halogen atom (for example, fluorine, chlorine, or bromine), a cyano group, a nitro group, and a hydroxyl group. The substituent may have further an additional substituent and examples of the additional substituent include the same as those denoted above.

In the above, R³⁵ is an amino group or a substituted amino group such as an alkylamino group, a dialkylamino group, an arylamino group, a diarylamino group, or an alkylarylamino group. The coumarin derivatives are preferably used in which the alkyl substituent of the substituted amino group in R³⁵ combines with R³⁴ or R³⁶ to form a ring.

In addition to the above, at least one of R³¹ and R³² is an alkyl group (for example, a methyl group, an ethyl group, a propyl group, an isopropyl group, a t-butyl group, a pentyl group, a hexyl group, an octyl group, a dodecyl group, a tridecyl group, a tetradecyl group, or a pentadecyl group), a cycloalkyl group (for example, a cyclopentyl group or a cyclohexyl group), an alkenyl group (for example, a vinyl group or an allyl group), an aryl group (for example, a phenyl group, or a naphthyl group), a heteroaryl group (for example, a furyl group, a thienyl group, a pyridazinyl group, a pyrimidinyl group, a pyrazinyl group, a triazinyl group, an imidazolyl group, a pyrazolyl group, a thiazolyl group, a benzimidazolyl group, a benzoxazolyl group, a quinazolyl group, or a phthalazinyl group), a saturated heterocyclic group (for example, a pyrrolidinyl group, an imidazolidinyl group, a morpholinyl group or an oxazolidinyl group), an alkoxycarbonyl group (for example, a methyloxycarbonyl group, an ethyloxycarbonyl group, a butyloxycarbonyl group, an octyloxycarbonyl group, or a dodecyloxycarbonyl group), an aryloxycarbonyl group (for example, a phenyloxycarbonyl group, or a naphthyloxycarbonyl group), an acyl group (for example, an acetyl group, an ethylcarbonyl group, a propylcarbonyl group, a pentylcarbonyl group, a cyclohexylcarbonyl group, an octylcarbonyl group, a 2-ethylhexylcarbonyl group, a dodecycarbonyl group, a phenylcarbonyl group, a naphthylcarbonyl group, or a pyridylcarbonyl group), an acyloxy group (for example, an acetyloxy group, an ethylcarbonyloxy group, a butylcarbonyloxy group, an octylcarbonyloxy group, a dodecycarbonyloxy group, or a phenylcarbonyloxy group), a carbamoyl group (for example, an aminocarbonyl group, a methylaminocarbonyl group, a dimethylaminocarbonyl group, a propylaminocarbonyl group, a pentylaminocarbonyl group, a cyclohexylaminocarbonyl group, an octylaminocarbonyl group, a 2-ethylhexylaminocarbonyl group, a dodecyaminocarbonyl group, a phenylaminocarbonyl group, a naphthylaminocarbonyl group, or a 2-pyridylaminocarbonyl group), a sulfinyl group (for example, a methylsulfinyl group, an ethylsulfinyl group, a butylsulfinyl group, a cyclohexylsulfinyl group, a 2-ethylhexylsulfinyl group, a dodecylsulfinyl group, a phenylsulfinyl group, a naphthylsulfinyl group, or a 2-pyridylsulfinyl group), an alkylsulfonyl group (for example, a methylsulfonyl group, an ethylsulfonyl group, a butylsulfonyl group, a cyclohexylsulfonyl group, a 2-ethylhexylsulfonyl group, or a dodecylsulfonyl group), an arylsulfonyl group (for example, a phenylsulfonyl group, a naphthylsulfonyl group, or a 2-pyridylsulfonyl group), a halogen atom (for example, fluorine, chlorine, or bromine), a cyano group, a nitro group or a halogenated alkyl group (for example, a trifluoromethyl group, a tribromomethyl group, or a trichloromethyl group).

Preferred examples of the dyes will be listed below, but the invention is not limited thereto.

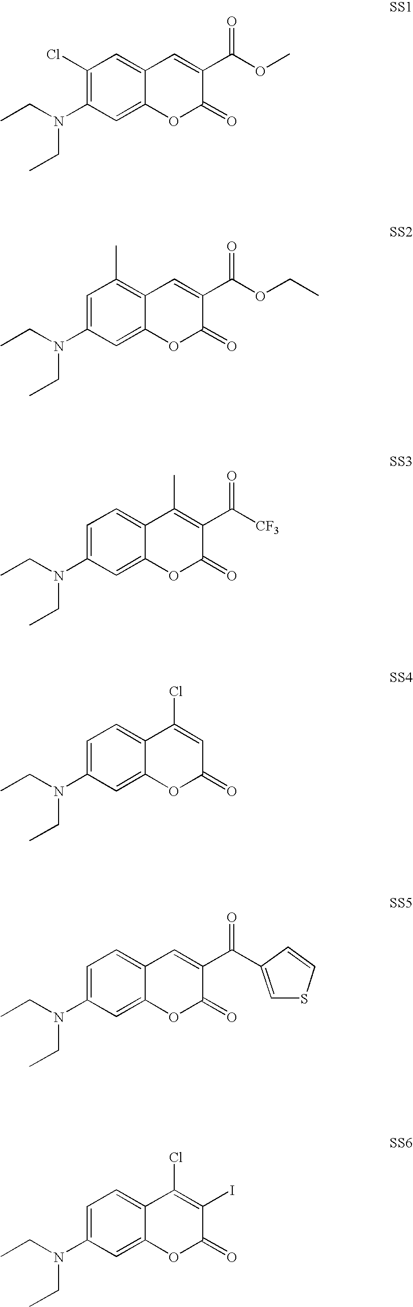

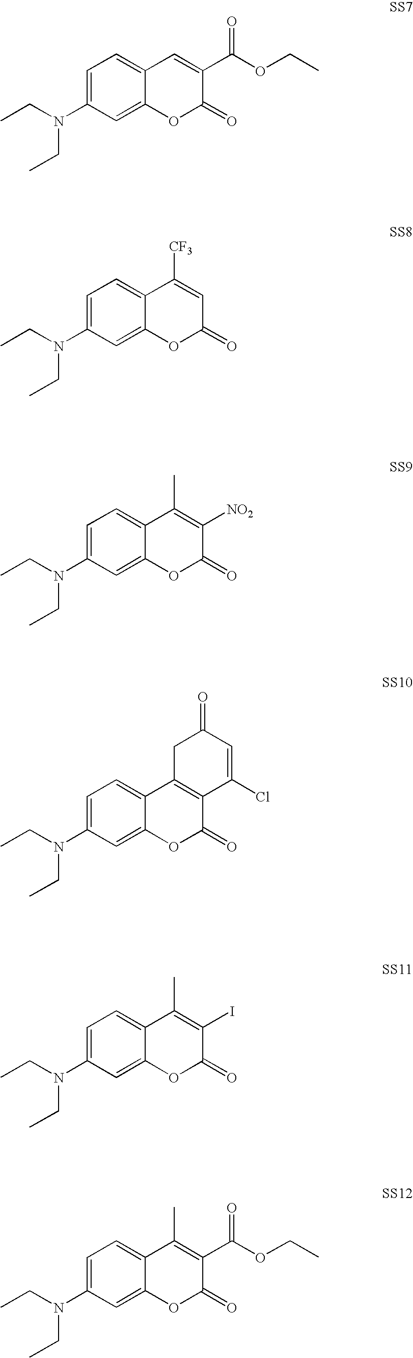

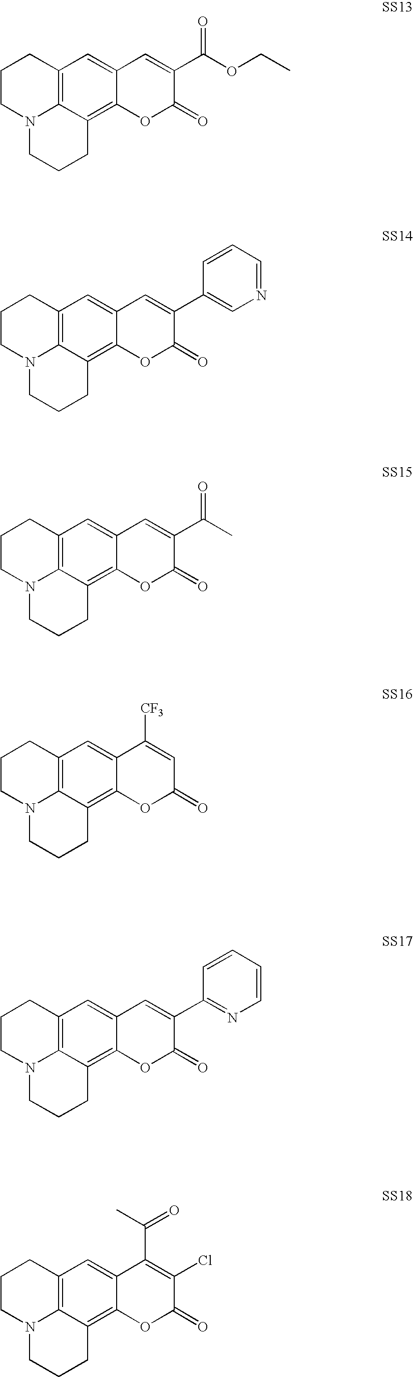

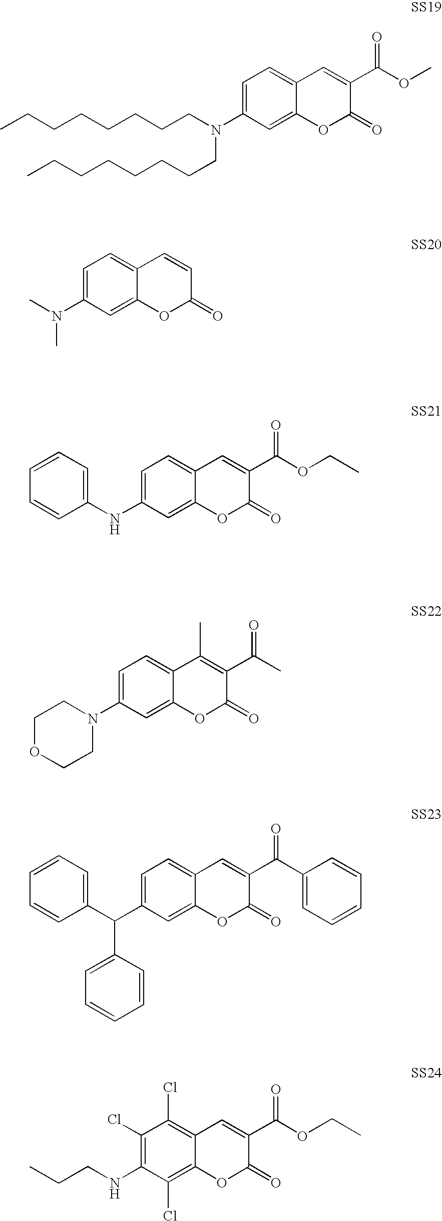

Besides the examples described above, there can be used coumarin derivatives B-1 through B-22 disclosed in Japanese Patent O.P.I. Publication No. 8-129258, coumarin derivatives D-1 through D-32 disclosed in Japanese Patent O.P.I. Publication No. 2003-12901, coumarin derivatives 1 through 21 disclosed in Japanese Patent O.P.I. Publication No. 2002-363206, coumarin derivatives 1 through 40 disclosed in Japanese Patent O.P.I. Publication No. 2002-363207, coumarin derivatives 1 through 34 disclosed in Japanese Patent O.P.I. Publication No. 2002-363208, or coumarin derivatives 1 through 56 disclosed in Japanese Patent O.P.I. Publication No. 2002-363209.

In the invention, a compound having absorption maximum absorbing at the same wavelength or approximately the same wavelength as the emission wavelength of light from a light source as described above is effective as a spectral sensitizing agent, and the effects of the invention are enhanced particularly when a spectral sensitizing agent adapted to near-infrared or infrared ray is employed.

### (Infrared Absorbing Agent)

In the invention, an infrared ray polymerization light sensitive layer is required to contain an infrared absorbing agent. The infrared absorbing agent in the invention is a compound having light absorption in the wavelength range of from 700 to 1200 nm. An infrared absorbing agent having light absorption in the wavelength range of from 700 to 1200 nm is not specifically limited. Preferred examples thereof include infrared absorbing agents, light-to-heat conversion materials, near-infrared dyes or pigments disclosed in U.S. Pat. No. 4,973572, Japanese Patent O.P.I. Publication Nos. 10-230582, 11-1538591, 2000-103179, 2000-187322, 2001-175006, 2002-537419, 2002-341519, 2003-76010, 2002-278057, 2003-5363, 2001-125260, 2002-23360, 2002-40638, 2002-62642, and 2002-2787057.

Cyanine dyes, squarylium dyes, oxonol dyes, pyrylium dyes, thiopyrylium dyes, polymethine dyes, oil-soluble phthalocyanine dyes, triarylamine dyes, thiazolium dyes, oxazolium dyes, polyaniline dyes, polypyrrole dyes and polythiophene dyes, can be preferably employed.

Among these, cyanine dyes, polymethine dyes and squarylium dyes are especially preferred.

The adding amount of the infrared absorbing agent in the invention in the light sensitive layer is arbitrary, but is preferably from 0.1 to 20% by weight, and more preferably from 0.8 to 15% by weight. Further, the adding amount of the infrared absorbing agent in the invention in the light sensitive layer is an amount such that absorption of reflection spectra is preferably 0.2 to 2.0, and more preferably 0.3 to 1.2, the reflection spectra being obtained according to integrating sphere measurement employing light with the wavelength of the laser used.

These infrared absorbing agents may be used singly or as an admixture of two or more kinds thereof.

Preferred examples of the infrared absorbing agents will be listed below.

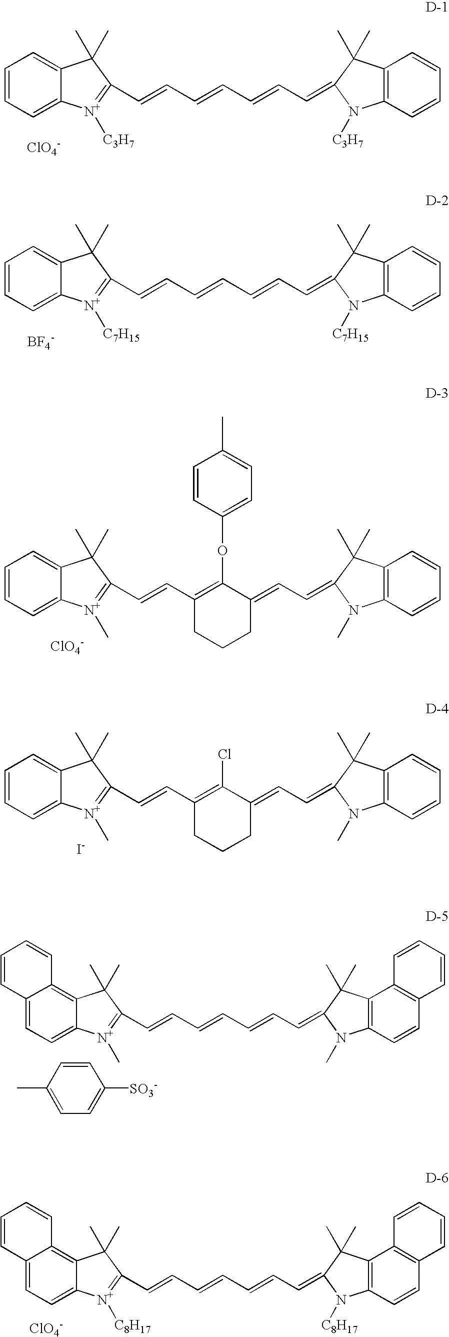

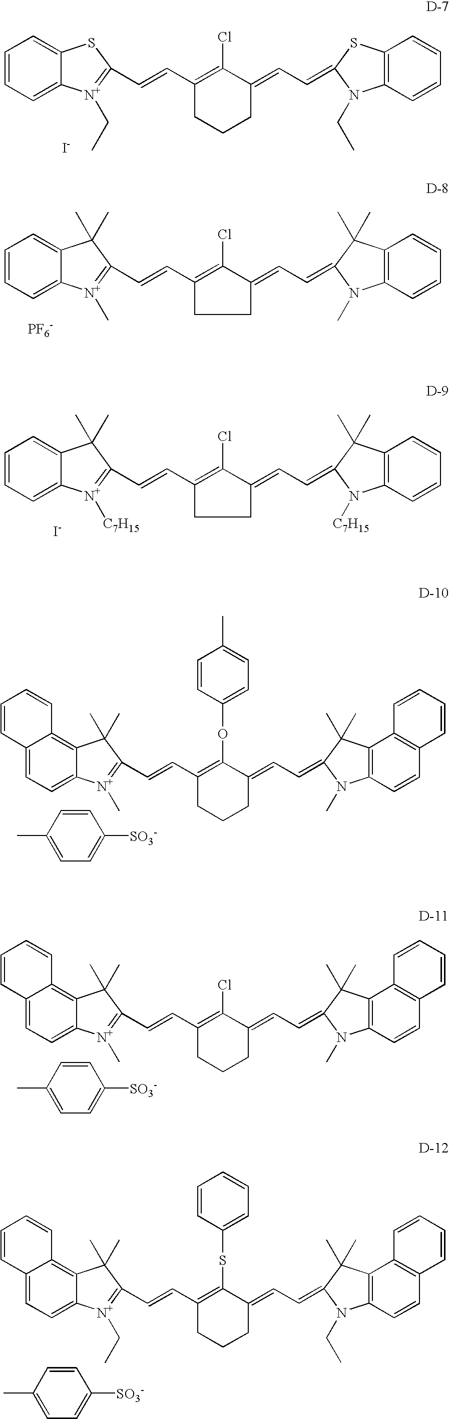

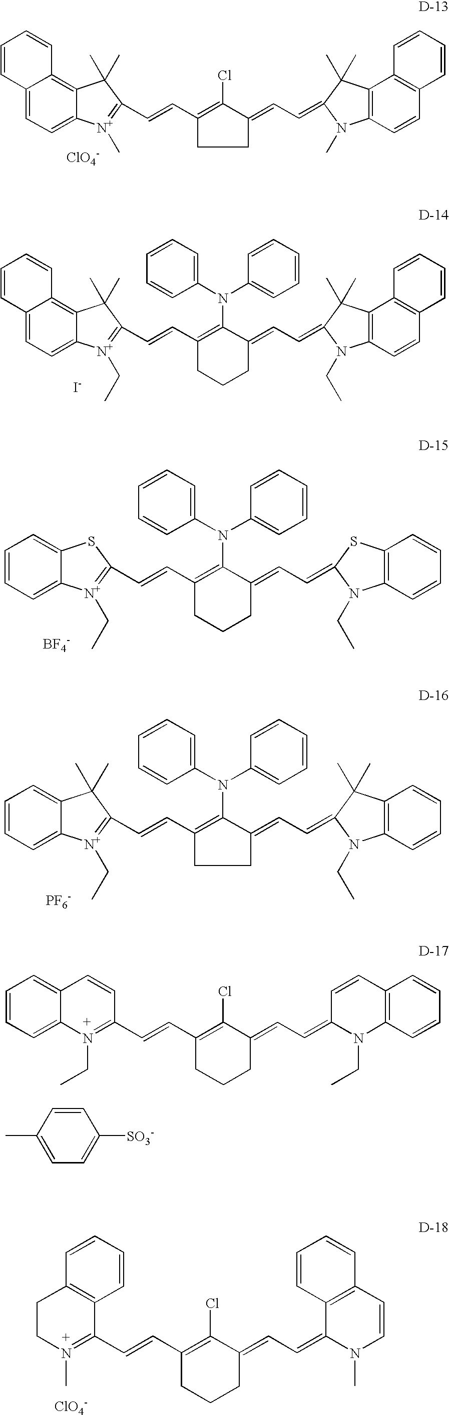

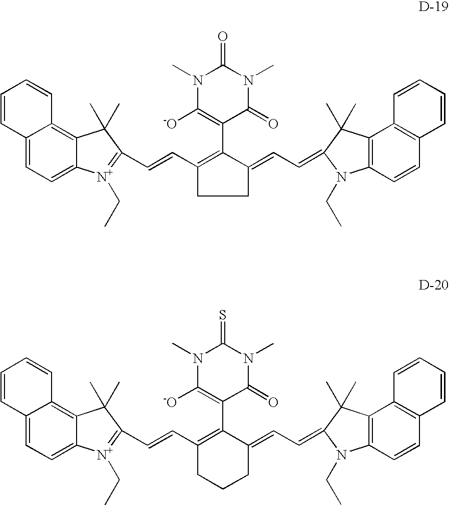

The adding amount of an infrared absorbing agent having absorption in the wavelength range of from 700 to 1200 nm in the light sensitive layer is different due to the extinction coefficient of the infrared absorbing agent and is preferably an amount such that the reflection density of a planographic printing plate material is preferably from 0.3 to 3.0, and more preferably from 0.5 to 2.0, measured at the wavelength of light for exposure. For example, when cyanine dyes as described above are used, the adding amount in the light sensitive layer which satisfies the above density is from 10 to 100 mg/m².

### (Various Additives)

The light sensitive layer in the invention may be added with a hindered phenol compound, a hindered amine compound or other polymerization inhibitors, in order to prevent undesired polymerization of the ethylenically unsaturated monomer during the manufacture or after storage of light sensitive planographic printing plate material.

Examples of the hindered phenol compound include 2,6-di-t-butyl-p-cresol, butylated hydroxyanisole, 2,2′-methylenebis(4-methyl-6-t-butylphenol), 4,4′-butylidenebis(3-methyl-6-t-butylphenol), tetrakis-[methylene-3-(3′5′-di-t-butyl-4′-hydroxyphenyl)propionate]methane, bis[3,3′-bis-(4′-hydroxy-3′-t-butylphenyl)butyric acid]glycol ester, 2-t-butyl-6-(3-t-butyl-2-hydroxy-5-methylbenzyl)-4-methylphenyl acrylate and 2-[1-(2-hydroxy-3,5-di-t-pentylphenyl)ethyl]-4,6-di-t-pentylphenyl acrylate. Preferred are 2-t-butyl-6-(3-t-butyl-2-hydroxy-5-methylbenzyl)-4-methylphenyl acrylate and 2-[1-(2-hydroxy-3,5-di-t-pentylphenyl)ethyl]-4,6-di-t-pentylphenyl acrylate, each having acrylate.

Examples of the hindered amine compound include bis(1,2,2,6,6-pentamethyl-4-piperidyl) sebacate, bis(2,2,6,6-tetramethyl-4-piperidyl) sebacate, 1-[2-[3-(3,5-di-t-butyl-hydroxyphenyl)propionyloxy]ethyl]-4-[3-(3,5-di-t-butyl-4-hydroxyphenyl)propionyloxy]-2,2,6,6-tetramethylpiperidine, 4-benzoyloxy-2,2,6,6-tetramethylpiperidine, and 8-acetyl-3-dodecyl-7,7,9,9-tetramethyl-1,3,8-triazaspiro[4.5]decane-2,4-dione.

Examples of other polymerization inhibitors include hydroquinone, p-methoxyphenol, di-t-butyl-p-cresol, pyrrogallol, t-butylcatechol, benzoquinone, 4,4′-thiobis(3-methyl-6-t-butylphenol), 2,2′-methylenebis(4-methyl-6-t-butylphenol), N-nitrosophenylhydroxylamine cerous salt, and hindered amines such as 2,2,6-6-tetramethylpieridine derivatives.

The polymerization inhibitor content is preferably 0.01 to 5% by weight based on the total solid content of the light sensitive layer. Further, in order to prevent undesired polymerization induced by oxygen, behenic acid or a higher fatty acid derivative such as behenic amide may be added to the layer. After the light sensitive layer is coated layer, the coated layer may be dried so that the higher fatty acid derivative is localized at the vicinity of the surface of the light sensitive layer. The content of the higher fatty acid derivative is preferably 0.5 to 10% by weight, based on the total solid content of the light sensitive composition.

The light sensitive layer in the invention can contain a colorant in addition to the components described above. As the colorant can be used known materials including commercially available materials. Examples of the colorant include those described in revised edition “Ganryo Binran”, edited by Nippon Ganryo Gijutu Kyoukai (published by Seibunndou Sinkosha), or “Color Index Binran”. Pigment is preferred.

Kinds of the pigment include black pigment, yellow pigment, red pigment, brown pigment, violet pigment, blue pigment, green pigment, fluorescent pigment, and metal powder pigment. Examples of the pigment include inorganic pigment (such as titanium dioxide, carbon black, graphite, zinc oxide, Prussian blue, cadmium sulfide, iron oxide, or chromate of lead, zinc, barium or calcium); and organic pigment (such as azo pigment, thioindigo pigment, anthraquinone pigment, anthanthrone pigment, triphenedioxazine pigment, vat dye pigment, phthalocyanine pigment or its derivative, or quinacridone pigment).

Among these pigment, pigment is preferably used which does not substantially have absorption in the absorption wavelength regions of a spectral sensitizing dye used according to a laser for exposure. The absorption of the pigment used is not more than 0.05, obtained from the reflection spectrum of the pigment measured employing an integrating sphere and employing light with the wavelength of the laser used.

The pigment content is preferably 0.1 to 10% by weight, and more preferably 0.2 to 5% by weight, based on the total solid content of light sensitive layer.

The light sensitive layer can contain a plasticizer in order to improve its adhesion to a support.

Examples of the plasticizer include dimethyl phthalate, diethyl phthalate, dibutyl phthalate, diheptyl phthalate, di-2-hexyl phthalate, di-n-octyl phthalate, didodecyl phthalate, dilsodecyl phthalate, butylbenzyl phthalate, diisononyl phthalate, ethylphtalylethyl phthalate, dimethyl isophthalate, triethylene glycol dicaprylate, dimethyl glycol phthalate, tricresyl phosphate, dioctyl adipate, dibutyl sebacate, and triacetyl glycerin. The plasticizer content is preferably from about 0 to 36 by weight, and more preferably from 0.1 to 2% by weight, based on the total solid content of composition.

A light sensitive layer to be coated can contain surfactants as a coating improving agent as long as the performance of the invention is not jeopardized. Among these surfactants, a fluorine-contained surfactant is preferred.

Further, in order to improve physical properties of the cured light sensitive layer, the light sensitive layer can contain an inorganic filler or a plasticizer such as dioctyl phthalate, dimethyl phthalate or tricresyl phosphate. The content thereof is preferably not more than 10% by weight based on the total solid content of light sensitive layer.

### (Coating)

The light sensitive planographic printing plate material of the invention is manufactured by preparing a light sensitive layer coating liquid containing the above-described components, then coating the light sensitive layer coating liquid on a support and drying.

The solvents used in the preparation of the light sensitive layer coating liquid include an alcohol such as sec-butanol, isobutanol, n-hexanol, benzyl alcohol, diethylene glycol, triethylene glycol, tetraethylene glycol, or 1,5-pentanediol; an ether such as propylene glycol monobutyl ether, dipropylene glycol monomethyl ether, or tripropylene glycol monomethyl ether; a ketone or aldehyde such as diacetone alcohol, cyclohexanone, or methyl cyclohexanone; and an ester such as ethyl lactate, butyl lactate, diethyl oxalate, or methyl benzoate.

Examples of the coating method include an air doctor coating method, a blade coating method, a wire bar coating method, a knife coating method, a dip coating method, a reverse roll coating method, a gravure coating method, a cast coating method, a curtain coating method, and an extrusion coating method.

### (Support)

The support used in the invention is a plate or a sheet capable of carrying the light sensitive layer and preferably has a hydrophilic surface on the side on which the light sensitive layer is to be provided.

As the support used in the invention, a plate of a metal such as aluminum, stainless steel, chromium or nickel, or a plastic film such as a polyester film, a polyethylene film or a polypropylene film, which is deposited or laminated with the above-described metal can be used.

Further, a polyester film, a polyvinyl chloride film or a nylon film whose surface is subjected to hydrophilization treatment can be used. Among the above, the aluminum plate is preferably used.

As the aluminum plate, a pure aluminum plate or an aluminum alloy plate is used.

As the aluminum alloy, there can be used various ones including an alloy of aluminum and a metal such as silicon, copper, manganese, magnesium, chromium, zinc, lead, bismuth, nickel, titanium, sodium or iron. In the aluminum plate for the support, the surface is roughened for water retention.

It is preferable that the aluminum plate is subjected to degreasing treatment for removing rolling oil prior to surface roughening (graining). The degreasing treatments include degreasing treatment employing solvents such as trichlene and thinner, and an emulsion degreasing treatment employing an emulsion such as kerosene or triethanol. It is also possible to use an aqueous alkali solution such as caustic soda for the degreasing treatment. When an aqueous alkali solution such as caustic soda is used for the degreasing treatment, it is possible to remove soils and an oxidized film which can not be removed by the above-mentioned degreasing treatment alone. When an aqueous alkali solution such as caustic soda is used for the degreasing treatment, the resulting support is preferably subjected to desmut treatment in an aqueous solution of an acid such as phosphoric acid, nitric acid, sulfuric acid, chromic acid, or a mixture thereof, since smut is produced on the surface of the support. The surface roughening methods include a mechanical surface roughening method and an electrolytic surface roughening method electrolytically etching the support surface.

Though there is no restriction for the mechanical surface roughening method, a brushing roughening method and a honing roughening method are preferable.

Though there is no restriction for the electrolytic surface roughening method, a method, in which the support is electrolytically surface roughened in an acidic electrolytic solution, is preferred.

After the support has been electrolytically surface roughened, it is preferably dipped in an acid or an aqueous alkali solution in order to remove aluminum dust, etc. produced in the surface of the support. Examples of the acid include sulfuric acid, persulfuric acid, hydrofluoric acid, phosphoric acid, nitric acid and hydrochloric acid, and examples of the alkali include sodium hydroxide and potassium hydroxide. Among those mentioned above, the aqueous alkali solution is preferably used. The dissolution amount of aluminum in the support surface is preferably 0.5 to 5 g/m². After the support has been dipped in the aqueous alkali solution, it is preferable for the support to be dipped in an acid such as phosphoric acid, nitric acid, sulfuric acid and chromic acid, or in a mixed acid thereof, for neutralization.

The mechanical surface roughening and electrolytic surface roughening may be carried out singly, and the mechanical surface roughening followed by the electrolytic surface roughening may be carried out.

After the surface roughening, anodizing treatment may be carried out. There is no restriction in particular for the method of anodizing treatment used in the invention, and known methods can be used. The anodizing treatment forms an anodization film on the surface of the support.

The support which has been subjected to anodizing treatment is optionally subjected to sealing treatment. For the sealing treatment, it is possible to use known methods using hot water, boiling water, steam, a sodium silicate solution, an aqueous dichromate solution, a nitrite solution and an ammonium acetate solution.

After the above treatment, the support is suitably undercoated with a water soluble resin such as polyvinyl phosphonic acid, a polymer or copolymer having a sulfonic acid in the side chain, or polyacrylic acid; a water soluble metal salt such as zinc borate; a yellow dye; an amine salt; and so on, for hydrophilization treatment. The sol-gel treatment support disclosed in Japanese Patent O.P.I. Publication No. 5-304358, which has a functional group capable of causing addition reaction by radicals as a covalent bond, is suitably used.

### (Protective Layer)

In the invention, a protective layer may be provided on the light sensitive layer.

Preferred materials constituting the protective layer are polyvinyl alcohol, polysaccharide, polyvinyl pyrrolidone, polyethylene glycol, gelatin, glue, casein, hydroxyethyl cellulose, carboxymethyl cellulose, methyl cellulose, hydroxyethyl starch, gum arabic, sucrose octacetate, ammonium alginate, sodium alginate, polyvinyl amine, polyethylene oxide, polystyrene sulfonic acid, polyacrylic acid, a water soluble polyamide and polyvinyl pyrrolidone derivatives. These can be used singly or as an admixture of two or more kinds thereof in the protective layer. Polyvinyl alcohol is especially preferred.

### (Imagewise Exposure; Plate Making Process)

As a light source for recording an image on the light sensitive planographic printing plate material of the invention, various ones can be employed according to the wavelength region to which the light sensitive layer is sensitive, and a laser light source is preferably used.

A printing plate can be prepared by exposing the light sensitive layer of the planographic printing plate material to laser according to image data to form an image.

When a laser is used for exposure, which can be condensed in the beam form, scanning exposure according to image data can be carried out, and direct writing is possible without using any mask material. When the laser is employed for imagewise exposure, a highly dissolved image can be obtained, since it is easy to condense its exposure spot in minute size.

### (Imagewise Exposure; Plate Making Process)

In the plate making process of the invention, imagewise exposure is carried out employing a laser light.

As a laser light source for exposing the light sensitive planographic printing plate material of the invention, a laser light source having an emission wavelength at the UV region and/or at the short visible wavelength region (350 to 450 nm) can be employed.

As the laser light source having an emission wavelength at the wavelength region of 350 to 450 nm which is available, there are a combination of a guiding wavelength conversion element and AlGaAs, InGaAs semiconductor (380 to 450 nm), a combination of a guiding wavelength conversion element and AlGaInP, InGaAs semiconductor (300 to 350 nm), AlGaInN (350 to 450 nm), a pulse laser such as an N₂ laser (337 nm, pulse: 0.1 to 10 mJ) or an XeF laser (351 nm, pulse: 10 to 250 mJ), a He—Cd laser (441 nm), a combination of Cr:LiSAF and SHG crystals (430 nm) as a solid laser, and KnbO₃, ring resonator (430 nm), AlGaInN (350 to 450 nm) or AlGaInN semiconductor laser (InGaN type semiconductor laser available on the market, 400 to 410 nm) as a semiconductor type laser.

As a laser light source for exposing the light sensitive planographic printing plate material of the invention, a laser light source having an emission wavelength at the infrared region and/or at the near-infrared region, i.e., at the wavelength region of 700 to 1500 nm, can be preferably employed. Typically, YAG laser or a semiconductor laser can be suitably employed.

As a laser scanning method of lasers used in the invention, there are a method of laser scanning on an outer surface of a cylinder, a method of laser scanning on an inner surface of a cylinder and a method of laser scanning on a plane. In the method of laser scanning on an outer surface of a cylinder, laser beam exposure is conducted while a drum around which a recording material is wound is rotated, in which main scanning is represented by the rotation of the drum, while sub-scanning is represented by the movement of the laser beam.

In the method of laser scanning on an inner surface of a cylinder, a recording material is fixed on the inner surface of a drum, a laser beam is emitted from the inside, and main scanning is carried out in the circumferential direction by rotating a part of or an entire part of an optical system, while sub-scanning is carried out in the axial direction by moving straight a part of or an entire part of the optical system in parallel with a shaft of the drum.

In the method of laser scanning on a plane, main scanning by means of a laser beam is carried out through a combination of a polygon mirror, a galvano mirror and an FO lens, and sub-scanning is carried out moving a recording medium. The cylinder outer surface laser scanning method and the cylinder inner surface laser scanning method are suitable for high density image recording, since it is easier to increase accuracy of an optical system.

In the invention, the exposed planographic printing plate material is preferably subjected to heat treatment before or during development. Such a heat treatment can increase adhesion between a support and an image formation layer provided thereon, resulting in increase of printing durability.

Regarding pre-heating treatment, there is, for example, a developing machine in which a preheating roller for preheating an exposed planographic printing plate material to a predetermined temperature is arranged upstream a development section where the preheating is carried out before development. The preheating roller is a roller comprised of a pair of rollers, at least one of the pair of the rollers having a heating means within the roller. The roller having a heating means in it is a pipe of a metal with high thermal conductivity such as aluminum or iron, the pipe having a nichrome wire as a heating element. The outside surface of the pipe may be covered with a sheet of a plastic such as polyethylene, polystyrene or Teflon. Details of such a preheating roller can refer to Japanese Patent O.P.I. Publication No. 64-80962.

In the invention, it is preferred that the preheating is carried out at 70 to 180° C. for 3 to 120 seconds.

### (Developer)

In the invention, the imagewise exposed image formation layer, which are cured are at exposed portions, is developed with an alkali developer, whereby the image formation layer at exposed portions are removed to form an image.

As the alkali developer, a conventional alkali aqueous solution is used. For example, there is an alkali developer containing an inorganic alkali agent such as sodium silicate, potassium silicate, ammonium silicate, sodium secondary phosphate, potassium secondary phosphate, ammonium secondary phosphate; sodium hydrogen carbonate, potassium hydrogen carbonate, ammonium hydrogen carbonate; sodium carbonate, potassium carbonate, ammonium carbonate; sodium borate, potassium borate, lithium borate; sodium hydroxide, potassium hydroxide, and ammonium hydroxide.

The alkali developer can contain organic alkali agents such as monomethylamine, dimethylamine, trimethylamine, monoethylamine, diethylamine, triethylamine, monoisopropylamine, diisopropylamine, triisopropylamine, n-butylamine, monoethanolamine, diethanolamine, triethanolamine, monoisopropanolamine, diisopropanolamine, ethyleneimine, ethylenediamine, and pyridine.

These alkali agents can be used singly or as a mixture of two or more thereof. The alkali developer can contain an anionic surfactant, an amphoteric surfactant, or an organic solvent such as alcohol.

### (Surfactant)

Various surfactants or organic solvents can be optionally added to a developer used in the invention, in order to accelerate development, disperse smuts occurring during development, or enhance ink receptivity at the image portions of a printing plate.

As the surfactant, there are an anionic surfactant, a cationic surfactant, a nonionic surfactant, and an amphoteric surfactant. Preferred examples of the nonionic surfactant include polyoxyethylenealkyl ethers, polyoxyethylenealkylphenyl ethers, polyoxyethylenepolystyrylphenyl ethers, polyoxyethylenepolyoxypropylenalkyl ethers, partial esters of glycerin and fatty acids, partial esters of sorbitan and fatty acids, partial esters of pentaerythritol and fatty acids, propylene glycol monofatty acid ester, partial esters of sucrose and fatty acids, partial esters of polyoxyethylenesorbitan and fatty acids, partial esters of polyoxyethylenesorbitol and fatty acids, esters of polyoxyethylene glycol and fatty acids, partial esters of polyglycerin and fatty acids, polyoxyethylene castor oil, partial esters of polyoxyethyleneglycerin and fatty acids, polyoxyethylene-polyoxypropylene block copolymer, adduct of polyoxyethylene-polyoxypropylene block copolymer with ethylene imine, fatty acid diethanolamides, N,N-bis-2-hydroxyalkylamines, polyoxyethylenealkylamines, triethanolamine fatty acid esters, and trialkylamine oxides.

Examples of the anionic surfactant include fatty acid salts, abietic acid salts, hydroxyalkane sulfonic acid salts, alkane sulfonic acid salts, dialkylsulfosuccinic acid salts, straight-chained alkylbenzene sulfonic acid salts, branched alkylbenzene sulfonic acid salts, alkylnaphthalene sulfonic acid salts, alkyldiphenylether sulfonic acid salts, alkylphenoxypolyoxyethylenepropyl sulfonic acid salts, polyoxyethylenealkyl sulfophenylether salts, N-methyl-N-oleiltaurine sodium salts, N-alkylsulfosuccinic acid monoamide disodium salts, petroleum sulfonic acid salts, nitrated castor oil, sulfated beef tallow, fatty acid alkyl ester sulfate salts, alkylsulfate salts, polyoxyethylenealkylethersulfate salts, fatty acid monoglyceride sulfate salts, polyoxyethylenealkylphenylethersulfate salts, polyoxyethylenestyrylphenylethersulfate salts, alkylphosphate salts, polyoxyethylenealkyletherphosphate salts, polyoxyethylenealkylphenyletherphosphate salts, partial saponification products of styrene-maleic anhydride copolymers, partial saponification products of olefin-maleic anhydride copolymers, and condensates of naphthalene sulfonic acid salts with formalin. Examples of the cationic surfactant include alkylamine salts, quaternary ammonium salts such as tetrabutylammonium bromide, polyoxyethylene alkylamine salts, and polyethylene polyamine derivatives. Examples of the amphoteric surfactant include carboxybetains, amino carboxylic acids, sulfobetaines, aminosulfates and imidazolines.

A more preferred surfactant is a fluorine-containing surfactant having a perfluoroalkyl group in the molecule. Examples thereof include anionic surfactants such as perfluoroalkylcarboxilic acid salts, or perfluoroalkylsurfuric acid salts, amphoteric surfactants such as perfluorobetaines, cationic surfactants such as perfluoroalkyltrimethylammonimum chlorides, and nonionic surfactants such as perfluoroalkylamineoxides, perfluoroalkylethyleneoxide adducts, oligomers having a perfluoroalkyl group and a hydrophilic group, oligomers having a perfluoroalkyl group and an oleophilic group, oligomers having a perfluoroalkyl group, a hydrophilic group, and an oleophilic group, and urethanes having a perfluoroalkyl group and an oleophilic group.

These surfactants can be used singly or as a mixture of two or more thereof. The developer preferably contains the surfactant in an amount of preferably from 0.001 to 100 by weight, and more preferably from 0.01 to 5% by weight.

### (Automatic Developing Machine)

The light sensitive planographic printing plate material is preferably developed through an automatic developing machine. It is preferred that a means for replenishing a developer replenisher in a necessary amount, a means for discharging any excessive developer, or a means for automatically replenishing water in necessary amounts is attached to the development section. It is preferred that the automatic developing machine comprises a means for detecting a transported planographic printing plate precursor, a means for calculating the area of the planographic printing plate precursor based on the detection, or a means for controlling the replenishing amount of a developer replenisher, the replenishing amount of water to be replenished, or the replenishing timing. It is also preferred that the automatic developing machine comprises a means for detecting a pH, temperature and/or electric conductivity of a developer, or a means for controlling the replenishing amount of the developer replenisher, the replenishing amount of water to be replenished or the replenishing timing, based on the detection. It is also preferred to provide a mechanism of diluting the developer concentrate with water and of stirring the diluted concentrate. Where the developing step is followed by a washing step, washing water used for washing can be reused as dilution water for diluting the developer concentrate.

The automatic developing machine used in the invention may be provided with a pre-processing section to allow the plate to be immersed in a pre-processing solution prior to development. The pre-processing section is provided preferably with a mechanism of spraying a pre-processing solution onto the plate surface, preferably with a mechanism of controlling the pre-processing solution at a temperature within the range of 25 to 55° C., and preferably with a mechanism of rubbing the plate surface with a roller-type brush. Common water and the like are employed as the pre-processing solution.

### (Post-Processing)

The developed printing plate material is preferably subjected to post-processing. The post-processing step comprises post-processing the developed precursor with a post-processing solution such as washing water, a rinsing solution containing a surfactant, a finisher or a protective gumming solution containing gum arabic or starch derivatives as a main component. The post-processing step is carried out employing an appropriate combination of the post-processing solution described above. For example, a method is preferred in which a developed planographic printing plate precursor is post-washed with washing water, and then processed with a rinsing solution containing a surfactant, or a developed planographic printing plate precursor is post-washed with washing water, and then processed with a finisher, since it reduces fatigue of the rinsing solution or the finisher. It is preferred that a multi-step countercurrent processing is carried out employing a rinsing solution or a finisher.

The post-processing is carried out employing an automatic developing machine having a development section and a post-processing section. In the post-processing step, the developed printing plate is sprayed with the post-processing solution from a spray nozzle or is immersed into the post-processing solution in a post-processing tank. A method is known in which supplies a small amount of water onto the developed printing plate precursor to wash the precursor, and reuses the water used for washing as a dilution water for developer concentrate. In the automatic developing machine, a method is applied in which each processing solution is replenished with the respective processing replenisher according to the area of the printing plate precursor to have been processed or the operating time of the machine. A method (use-and-discard method) can be applied in which the developed printing plate material is processed with fresh processing solution and discarded. The thus obtained planographic printing plate is mounted on a printing press, and printing is carried out.

### (Gum Solution)

Gum solution may be suitably added with acids or buffers to remove from the developed plate alkaline ingredients which are contained in the developer. Further, there may be added a hydrophilic polymer compound, a chelating agent, a lubricant, an antiseptic and a solubilizing agent. Inclusion of the hydrophilic polymer compound in the gum solution provides a function as a protecting agent to prevent the developed plate from flawing or staining.

### (Washing Water Used Prior to Development)

Washing solution used in the washing stage prior to development invention is usually common water and may optionally be added with additives such as chelating agents, surfactants or antiseptics.

In washing, a washing solution used prior to development is used preferably at a controlled temperature, and more preferably at 10 to 60° C. Washing can be performed using commonly known solution-feeding techniques such as a spraying, dipping or coating method. During washing, a wash promoting method employing a brush, a squeezing roll or a submerged shower in a dipping treatment can be suitably used.

After completion of the washing stage prior to development, development may be immediately conducted, or drying may be conducted after the washing stage, subsequently, development may be performed. The development stage is followed by a post-treatment such as washing, rinsing or a gumming treatment. Washing water used prior to development may also be reused as washing water or for a rinsing solution or gumming solution.

### (On-Press Development)

In the invention, one of the preferred embodiment is a printing process employing on-press development. The light sensitive planographic printing plate material of the invention comprising an infrared ray polymerization light sensitive layer in which image is formed by infrared laser is preferably subjected to on-press development. In the printing process of the invention, an exposed planographic printing plate material is mounted on a press without any wet processing, and developed by supplying dampening water and/or printing ink on the press to form an image, followed by printing.

Imagewise exposure is carried out before a planographic printing plate material is mounted on a press, or is carried out after a planographic printing plate material is mounted on a press, followed by printing.

In the invention, on-press development means that a planographic printing plate material on a printing press is supplied with dampening water and/or printing ink prior to printing to remove the non-image portions, that is, development is carried out on a printing press.

Removal (on-press development) of the image formation layer at non-image portions (unexposed portions) of an exposed printing plate material mounted on the plate cylinder of a printing press, is carried out by bringing a dampening roller or both of a dampening roller and an inking roller into contact with the image formation layer of the exposed printing plate material while rotating the plate cylinder to supply dampening water or both dampening water or a printing ink to the image formation layer.

The on-press development can be carried out, for example by various sequences as described below or another appropriate sequence. The amount of dampening water supplied during on-press development may be adjusted to be greater or smaller than the amount of the dampening water ordinarily supplied in printing, and the adjustment may be carried out stepwise or continuously.

Sequence (1) A dampening roller is brought into contact with the image formation layer of a printing plate material on the plate cylinder while one to several tens of rotations of the plate cylinder are carried out, and then an inking roller brought into contact with the image formation layer while one to tens of rotations of the plate cylinder are carried out. Thereafter, printing is carried out.

Sequence (2) An inking roller is brought into contact with the image formation layer of a printing plate material on the plate cylinder while one to several tens of rotations of the plate cylinder are carried out, and then a dampening roller brought into contact with the image formation layer while one to tens of rotations of the plate cylinder are carried out. Thereafter, printing is carried out.

Sequence (3) An inking roller and a dampening roller are simultaneously brought into contact with the image formation layer of a printing plate material on the plate cylinder while one to several tens of rotations of the plate cylinder are carried out. Thereafter, printing starts.

Known planographic offset printing presses are generally employed as a printing press.

### (Dampening Water)

Dampening water generally applied for printing is usable as the dampening water. Dampening water comprised only of water or a dampening water containing additives may be used.

Dampening water containing no isopropanol, which is conventionally used, is preferably used as dampening water.

In this case, dampening water not containing isopropanol means one having an isopropanol content of less than 0.5% by weight.

An aqueous solution containing a surfactant is preferably used as the dampening water.

As water for dampening water, water such as tap water or well water or so forth can be utilized.

The dampening water may contain a slight amount of components, such as acids, for example, a phosphoric acid or its salt, a citric acid or its salt, a nitric acid or its salt and an acetic acid or its salt, specifically, phosphoric acid, ammonium phosphate, sodium phosphate, citric acid, ammonium citrate, sodium citrate, acetic acid, ammonium acetate or sodium acetate, or water-soluble polymeric compounds such as carboxymethylcellulose, carboxyethylcellulose and so forth.

The content of these components is less than 0.1% by weight, and preferably less than 0.05% by weight.

Further, the dampening water may contain glycol-based compounds such as propylene glycol monomethylether, propylene glycol monoethylether, propylene glycol monobutylether, propylene glycol propyl ether, propylene glycol dimethyl ether, propylene glycol diethyl ether, propylene glycol dibutyl ether, dipropylene glycol dimethyl ether, dipropylene glycol diethyl ether, dipropylene glycol dibutyl ether and so forth. The content of the glycol-based compound is preferably a small amount of less than 0.1% by weight, and more preferably less than 0.05% by weight.

The dampening water may contain a surfactant.

A nonionic surfactant, an anionic surfactant, a cationic surfactant or a mixture thereof is preferably used as the surfactant.

Examples of the anionic surfactant include fatty acid salts, abietic acid salts, hydroxyalkane sulfonic acid salts, alkane sulfonic acid salts, dialkylsulfosuccinic acid salts, straight-chained alkylbenzene sulfonic acid salts, alkylnaphthalene sulfonic acid salts, alkylphenoxypolyoxyethylenepropyl sulfonic acid salts, polyoxyethylenealkyl sulfophenylether salts, N-methyl-N-oleiltaurine sodium salts, N-alkylsulfosuccinic acid monoamide disodium salts, petroleum sulfonic acid salts, nitrated castor oil, sulfated beef tallow, fatty acid alkyl ester sulfate salts, polyoxyethylenealkylethersulfate salts, fatty acid monoglyceride sulfate salts, polyoxyethylenealkylphenylethersulfate salts, polyoxyethylenestyrylphenylethersulfate salts, alkylphosphate salts, polyoxyethylenealkyletherphosphate salts, polyoxyethylenealkylphenyletherphosphate salts, partial saponification products of styrene-maleic anhydride copolymers, and condensates of naphthalene sulfonic acid salts with formalin.

Examples of the nonionic surfactant include polyoxyethylenealkyl ethers, polyoxyethylenealkylphenyl ethers, polyoxyethylene-polystyrylphenyl ethers, polyoxyethylenepolyoxypropylenalkyl ethers, partial esters of glycerin and fatty acids, partial esters of sorbitan and fatty acids, partial esters of pentaerythritol and fatty acids, propylene glycol monofatty acid ester, partial esters of sucrose and fatty acids, partial esters of polyoxyethylenesorbitan and fatty acids, partial esters of polyoxyethylenesorbitol and fatty acids, esters of polyoxyethylene glycol and fatty acids, partial esters of polyglycerin and fatty acids, polyoxyethylene castor oil, partial esters of polyoxyethyleneglycerin and fatty acids, fatty acid diethanolamides, N,N-bis-2-hydroxyalkylamines, polyoxyethylenealkylamines, triethanolamine fatty acid esters, trialkylamine oxides, and polyoxyethylene-polyoxypropylene block copolymer. Besides the above, a fluorine-contained surfactant or a silicon-contained surfactant can be employed.

Examples of the cationic surfactant include alkylamine salts, quaternary ammonium salts polyoxyethylene alkylamine salts, and polyethylene polyamine derivatives.

These surfactants may be used singly or as admixture of two or more kinds thereof. The surfactant content of the dampening water is preferably not more than 0.01% by weight, and more preferably not more than 0.05% by weight.

### (Printing)

A planographic printing plate prepared via the printing plate preparing process of the present invention is subjected to printing, but a conventional planographic offset printing press in which dampening water is utilized as described below is usable as a printing press. Printing paper, printing ink, dampening water and so forth which are used for printing are not specifically limited.

In recent years, printing ink containing no petroleum volatile organic compound (VOC) has been developed and used in view of environmental concern. The present invention provides excellent effects in employing such a printing ink friendly to environment.

As the ink, ink containing soybean oil is preferable.

Generally, the ink containing soybean oil is one mixed with an organic pigment, an inorganic pigment, a binder resin, soybean oil or a high boiling point petroleum solvent, and may also contain a plasticizer, a stabilizer, a desiccant agent, a thickner, a dispersant, a filler or such as an auxiliary substance.

As ink preferably usable for printing, ink accredited via the soy seal recognition system by American Soybean Association (ASA) is provided.

Commonly known soybean oil is usable as the soybean oil, and soybean oil for food (purified soybean oil) accredited by Japan Agricultural Standards.

The preferred soybean oil content of printing ink is different due to kinds of printing ink. The soybean oil content of printing ink for sheet-feed printing or form printing is from 20 to 50% by weight, that of heat-set printing ink for offset printing from 7 to 20% by weight, that of black printing ink for news paper printing from 40 to 50% by weight, that of color printing ink for news paper printing from 30 to 40% by weight, and that of printing ink for business form printing from 20 to 30% by weight.

As inorganic or organic pigments, bisazo yellow, brilliant carmine 6B, phthalocyanine blue, lake red C, carbon black, titanium oxide and calcium carbonate can be employed.

The binder resin is preferably natural or processed resin such as rosin, copal, dammar, shellac, cured rosin or rosin ester; phenol resin; rosin-modified phenol resin; 100% phenol resin; maleic acid resin; alkyd resin; petroleum resin; vinyl resin; acryl resin; polyamide resin; epoxy resin; aminoalkyd resin; polyurethane resin; or aminoblast resin.

The ink containing soybean oil is sold by various ink manufacturers, and easily purchased. Examples thereof include Naturalith 100 sheet-fed printing ink and WebWorldAdvan web offset printing ink (produced by Dainippon Ink and Chemicals, Inc.), TK Hyunity SOY sheet-fed printing ink, TK HyEcho-SOY sheet-fed printing ink, CK WINECO-SOY sheet-fed printing ink, WD Super LeoEcoo-SOY web offset printing ink, WD LeoEcoo-SOY web offset printing ink and SCRSOY Business Form ink (produced by Toyo Ink MFG. Co., Ltd.), and Soycelvo sheet-fed printing ink (produced by Tokyo Printing Ink MFG. Co., Ltd.).

### EXAMPLES

Next, the present invention will be explained below employing examples, but the present invention is not limited thereto. In the examples, “parts” represents “parts by weight”, unless otherwise specified.

### Example 1

### Preparation of Support

A 0.30 mm thick aluminum plate (material 1052, containing not less than 99.3% of Al, 0.003% of Na, 0.20% of Mg, 0.08% of Si, 0.06% of Ti, 0.004% of Mn, 0.32% of Fe, 0.004% of Ni, 0.002% of Cu, 0.015% of Zn, 0.007% of Ga and 0.001% of Cr) was degreased at 55° C. for 15 seconds in a 5% sodium hydroxide solution, and washed with water.

The degreased aluminum plate was immersed at 25° C. for 10 seconds in a 10% nitric acid solution to neutralize, and then washed with water.

The resulting aluminum plate was electrolytically surface-roughened using a 60 Hz sine wave alternating current source, and desmutted at 60° C. for 10 seconds in a 100 g/liter phosphoric acid solution, followed by washing with water.

The desmutted aluminum plate was anodized at a current density of 10 A/dm² in a 350° C. sulfuric acid (200 g/liter) solution through a direct current source to form an anodization layer of 20 mg/dm².

The resulting anodized aluminum plate was immersed in a 0.44% polyvinyl phosphonic acid aqueous solution at 75° C. for 30 seconds, washed with distilled water, and dried to obtain a support.

The center line average surface roughness (R^{a}) of the support was 0.45 μm.

### (Synthesis of Polymer Binder)

### Synthesis of Monomer (SM-1)

2,5-Dimercapto-13,4-thiadiazole (bismuthiol, produced by Tokyo Kasei Kogyo Co., Ltd.) of 357.6 g (2.38 mol) were suspended in 1.2 kg of methanol, and 234.0 g (2.31 mol) of triethylamine (produced by Nacarai Tesque Co., Ltd.) were added thereto over a period of 20 minutes with stirring while cooling with water. To the resulting yellow solution were dropwise added 305.2 g (2.00 mol) of 4-chloromethylstyrene (CMS-14, produced by Seimi Chemical Co., Ltd.) over a period of one hour with stirring while cooling with water, and further stirred for 2 hours at the same temperature. After that, the resulting precipitated crystals were filtered off, repeatedly washed with methanol, and dried to obtain 357.0 g (yield: 67%) of yellow-white crystal, Monomer (SM-1) having the following chemical structure, i.e., 2-(4-vinylbenzylthio)-5-mercapto-1,3,4-thiadiazole.

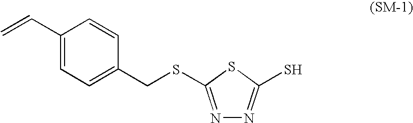

### Synthesis of Intermediate Polymer (SM-2)

The monomer (SM-1) obtained above of 66.4 g (1.00 mol) were suspended in a mixture of 710.4 g of ethanol and 177.6 g of ion-exchange water and 121.5 g (1.20 mol) of triethylamine

(produced by Nacarai Tesque Co., Ltd.) were added thereto over a period of 20 minutes at room temperature with stirring. The resulting yellow solution was heated on hot-water bath with stirring under nitrogen atmosphere to 75° C. Subsequently, 2.0 g of 2,2′-azobisisobutyronitrile (produced by Tokyo Kasei Kogyo Co., Ltd.) were added thereto at the same temperature to initiate polymerization. The polymerization was carried out for 8 hours, and a yellow solution containing Intermediate polymer (SM-2) having the following chemical structure was obtained. The solution was added with 2.0 g of N-nitrosophenylhydroxylamine ammonium salt (Q-1300, produced by Wako Junyaku Kogyo Co., Ltd.) as a polymerization inhibitor. The resulting solution was used in the following reaction for synthesis of Polymer (SP-1).

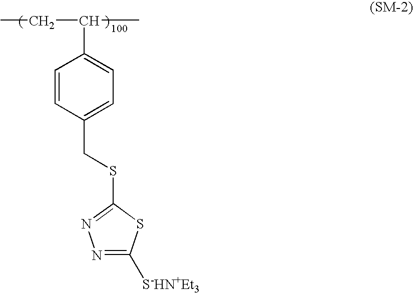

### Synthesis of Polymer (SP-1)

The solution containing Intermediate polymer (SM-2) obtained above was heated on hot-water bath with stirring to 50° C., added with 73.3 g (0.60 mol) of propanesultone (produced by Tokyo Kasei Kogyo Co., Ltd.) at the same temperature, and reacted for 3 hours. Subsequently, 73.3 g (0.48 mol) of 4-chloromethylstyrene (CMS-14, produced by Seimi Chemical Co., Ltd.) were added, and reacted for another 3 hours. After that, the resulting reaction solution was poured into 1.0 kg of diisopropyl ether to produce a light yellow polymer. After decantation of the supernatant, the residual polymer was dissolved in methanol, and re-precipitated in 1.0 kg of diisopropyl ether. The resulting re-precipitated polymer was sufficiently washed with acetone, added with 2.0 g of N-nitrosophenylhydroxylamine ammonium salt (Q-1300, produced by Wako Junyaku Kogyo Co., Ltd.) as a polymerization inhibitor, and dissolved in methanol to obtain a total amount of 1.5 kg. Thus, a methanol solution of Polymer (SP-1) was obtained. The solid content of this methanol solution was 29%. The yield of Polymer (SP-1) was 435 g (97%). The weight average molecular weight of Polymer (SP-1) was 65000 (in terms of polystyrene), measured according to gel permeation chromatography.

### Synthesis of Polymerizable Monomer

Polymerizable ethylenically unsaturated compound (hereinafter referred to as polymerizable monomer) (PM-1) or (PM-2) was synthesized employing, as raw material, butyldiethanolamine, hexamethylene diisocyanate, hydroxyethyl methacrylate or diethylene glycol as shown in Table 1. The synthesis was carried out according to the method disclosed in U.S. Pat. No. 2,669,849.

TABLE 1
(Composition of polymerizable monomer)

| | | | OH— containing | |
|---|---|---|---|---|
| | Tertiary amine diol | Diisocyanate | Acrylate | |
| | Unit A | Unit I | Unit R | Alkylene diol Unit O |
| PM-1 | Butyldiethanolamine | Hexamethylene | Hydroxyethyl | |
| (three component type) | | Diisocyanate | methacrylate | |
| Molar ratio | 1 | 2 | 2 | |
| PM-2 | Butyldiethanolamine | Hexamethylene | Hydroxyethyl | Diethylene glycol |
| (four component type) | | Diisocyanate | methacrylate | |
| Molar ratio | 1 | 4 | 2 | 2 |

Three component type R-I-A-I-R
Four component type R-I-O-I-A-I-O-I-R
PM-1 (Three component type)

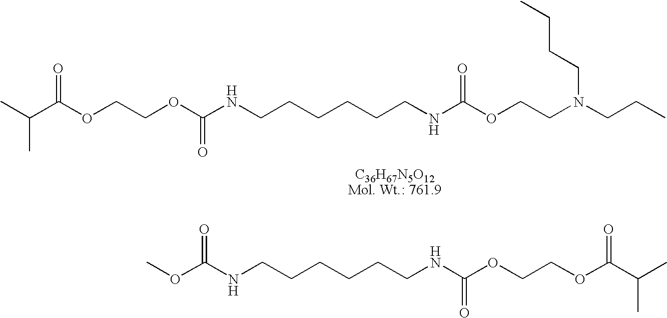

PM-2 (Four component type)

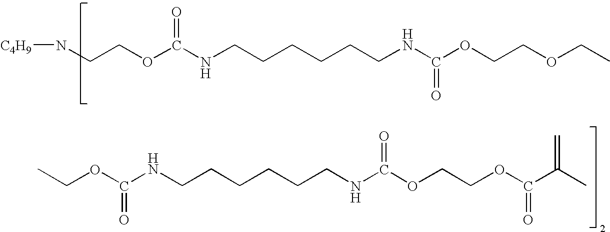

### (Preparation of Light Sensitive Planographic Printing Plate Material Samples)

The following photopolymerizable light sensitive layer coating solution was coated on the resulting support through a wire bar, and dried at 95° C. for 1.5 minutes to give a photopolymerizable light sensitive layer having a dry coating amount of 1.5 g/m².

Subsequently, the following oxygen shielding layer coating solution was coated on the resulting photopolymerizable light sensitive layer using a wire bar, and dried at 75° C. for 1.5 minutes to form an oxygen shielding layer with a dry coating amount of 0.7 g/m². Thus, light sensitive printing plate material samples (as shown in Table 2) with an oxygen shielding layer on a light sensitive layer were prepared.

(Photopolymerizable light sensitive layer coating solution 1)

| Polymer binder 1 (shown in Table 2) | 45.0 | parts |
|---|---|---|
| Polymerizable monomer (shown in Table 2) | 45.0 | parts |
| Infrared absorbing agent (Cyanine dye described later) | 5.0 | parts |
| Polyhalogen compound (Polymerization initiator-1 described later) | 5.0 | parts |
| Phthalocyanine pigment (MHI8974M produced by Mikuni Sikisosha) | 0.5 | parts |
| 2-t-Butyl-6-(3-t-butyl-2-hydroxy-5-methylbenzyl)-4-methylphenyl acrylate | 0.5 | parts |
| (Sumirizer GS: produced by Sumitomo 3M Co., Ltd.) | | |
| Surfinol 465 (available from Air Products Co., Ltd.) | 0.5 | parts |
| Methanol | 75 | parts |
| Ethanol | 75 | parts |
| Water (Protective layer coating solution) | 850 | parts |
| Polyvinyl alcohol (AL-06, produced by Nippon Gosei Kagaku Co., Ltd.) | 95 | parts |
| Vinyl pyrrolidone copolymer (VA64W, produced by BASF Inc.) | 5 | parts |
| Surfactant (Surfinol 465, available from Nisshin Kagaku Co., Ltd.) | 0.5 | parts |
| Water | 900 | parts |
| Cyanine dye-1 | | |
| 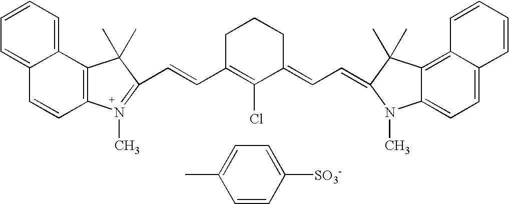 | | |
| Polymerization initiator-1 | | |
| 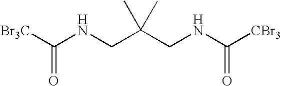 | | |

### (Evaluation of Printing Durability)

### (Image Formation Employing Infrared Laser)

The resulting planographic printing plate material sample was imagewise exposed employing an infrared laser.

Employing a 808 nm laser with a beam spot diameter of about 18 μm, the planographic printing plate material sample was imagewise exposed at exposure energy of from 100 to 450 mJ/cm² to form an image with a resolution of 2,400 dpi (dpi means a dot number per 2.54 cm) and a screen line number of 175, the exposure energy being changed at an interval of 50 mJ/cm².

The resulting sample was developed with a 25° C. tap water while rubbing with a sponge for PS plates to obtain a planographic printing plate sample with a developed image.

### (Sensitivity)

Each of the planographic printing plate material samples was processed as above to form a developed image.

Density of the developed image at each exposure energy level was measured through a densitometer D196 (produced by GRETAG Co., Ltd.).

An exposure energy at which optical density begins decreasing from the saturated solid image density was defined as sensitivity. Sensitivity was represented in terms of energy giving the saturated solid image density×0.9.

### (Printing Durability)

The resulting planographic printing plate sample being mounted on a press (DAIYA1F-1 produced by Mitsubishi Jukogyo Co., Ltd.), printing was carried out wherein a coat paper, printing ink (Soybean oil ink, NCP Naturalith produced by Dainippon Kagaku Kogyo Co., Ltd.), and dampening water (SG-51, H solution produced by Tokyo Ink Co., Ltd., Concentration: 1.5%) were used. Every time one hundred prints were printed, the printing plate surface was cleaned with a cleaner. The number of prints, which were printed until dot reduction at highlight portions of the print or filling-up (adhesion of printing ink to non-image portions around dot images) at shadow portions of the print was observed, was determined as a measure of printing durability.

The more the number is, the higher the printing durability.

Ultra Plate Cleaner (produced by Dainichi Seika Co., Ltd.) was employed as the cleaner.

### (Evaluation of On-Press Developability)

An optimum exposure amount (exposure energy three times exposure energy providing sensitivity) was calculated from the image formation method and sensitivity measurement described above.

A sample imagewise exposed at the optimum exposure energy was mounted without water development on the same printing press as used above in evaluation of printing durability, printing was started in a conventional procedure. Five hundred prints were printed, and every time one hundred prints were printed, a printed sample was taken.

Contamination at non-image portions and dot reproduction of the printed sample taken were visually observed, and the number of prints printed until the prints with good image quality were obtained was determined and evaluated.

The results are collectively shown in Table 2.

TABLE 2

| | | | | | Solubility | |
|---|---|---|---|---|---|---|
| | | | | | of | |
| | | | | | polymerizable | |
| Kind of | Kind of | | | | monomer | |
| polymer | polymerizable | Sensitivity | Printing | On-press | weight % | |
| binder | monomer | μj/cm² | durability | Developability | (water) | Remarks |
| PVP | PETA | 300 | 10,000 | 500 or | 0.1 | Comp. |
| | | | | more | | |
| PVP | PM-1 | 200 | 80,000 | 300 | 0.9 | Inv. |
| PVP | PM-1 | 200 | 80,000 | 100 | 2 | Inv. |
| SP-1 | PM-2 | 200 | 100,000 | 200 | 0.9 | Inv. |
| SP-1 | PM-2 | 200 | 100,000 | 100 | 2 | Inv. |
| SP-1 | PETA | 300 | 15,000 | 500 | 0.1 | Comp. |

PVP: Vinylpyrrolidone homopolymer (Luvitek produced by BASF)
PETA: Pentaerythritol triacrylate

As is apparent from Table 2, inventive samples excel in sensitivity, printing durability, and on-press developability, and the polymerizable monomer used in inventive samples excel in solubility.

### Example 2

Polymer binder (SP-2) was prepared as described below, and light sensitive planographic printing plate material samples as described below and as shown in Table 3 were prepared.

### (Preparation of Polymer Binder (SP-2))

Methyl methacrylate of 70.0 parts, 30.0 parts of methacrylic acid, 100 parts of ethanol, and 1.23 parts of α,α′-azobisisobutylonitrile were placed in a three neck flask under nitrogen atmosphere, and reacted under nitrogen atmosphere for 6 hours at 80° C. in an oil bath. Thus, polymer binder (SP-2) was prepared. The resulting polymer binder had a weight average molecular weight of 30,000, measured according to GPC, and had an acid value of 190 and a Tg of 142° C.

### (Preparation of Light Sensitive Planographic Printing Plate Material Samples)

The following photopolymerizable light sensitive layer coating solution was coated on the resulting support through a wire bar, and dried at 90° C. for 1.5 minutes to give a light sensitive layer having a dry coating amount of 1.7 g/m².

Subsequently, the following protective layer coating solution was coated on the resulting light sensitive layer using an applicator, and dried at 100° C. for 1.5 minutes to form a protective layer with a dry coating amount of 1.7 g/m². Thus, light sensitive printing plate material samples with a protective layer on a light sensitive layer were prepared.

(Photopolymerizable light sensitive layer coating solution)

| | |
|---|---|
| Polymer binder (shown in Table 3) | 45.0 parts |
| Spectral sensitizing agent (SS7) | 4.0 parts |
| Initiator (Compound as shown in Table 3) | 3.2 parts |
| Co-initiator* (Compound as shown in Table 3) | 2.5 parts |
| Polymerizable monomer (as shown in Table 3) | 37.0 parts |
| Polyethylene glycol #200 dimethacrylate | 8.0 parts |
| (NK ESTER4G, produced by ShinNakamura Kagaku | |
| Kogyol Co., Ltd.) | |
| Phthalocyanine pigment | 3.0 parts |
| (MHI454 produced by Mikuni Sikisosha, 30% MEK | |
| dispersion) | |
| Hindered amine light stabilizer | 0.5 parts |
| (LS770 produced by Sankyo Life-Tech Co., Ltd.) | |
| Fluorine-contained surfactant | 0.1 parts |
| (F178K produced by Dainippon ink Kagaku Kogyo Co., | |
| Ltd.) | |
| Cyclohexanone (bp. 155° C.) | 820 parts |

*Note: “Co-initiator” is a compound having a function capable of enhancing radical generation efficiency of an initiator. Co-initiator 1 is the same as the polymerization initiator used in Example 1. In Example 2, a combination of the initiator and the co-initiator forms a high sensitivity initiating system.

(Protective layer coating solution)

| | |
|---|---|
| Polyvinyl alcohol NL06 | 70 parts |
| (produced by Nippon Gosei Kagaku Co., Ltd.) | |
| Vinyl pyrrolidone copolymer (VA64W, | 30 parts |
| produced by BASF Inc.) | |
| Surfactant | 0.5 parts |
| (Surfinol 465, available from Nisshin Kagaku Co., Ltd.) | |
| Water | 900 parts |

### (Image Formation)

Each of the photopolymerization type planographic printing plate material samples obtained above was imagewise exposed at a resolution of 2400 dpi, employing a plate setter (News CTP produced by ECRM Co., Ltd.) equipped with a 408 nm laser with an output power of 60 mW. Herein, dpi represents the dot numbers per 2.54 cm.

Subsequently, the exposed sample was subjected to development treatment employing a CTP automatic developing machine (Raptor Polymer 85 produced by G & J Co., Ltd.) to obtain a planographic printing plate. Herein, the developing machine comprised a heating section for heating the exposed sample before development, a pre-washing section for removing the protective layer before development, a development section charged with developer having the following developer composition 1, a washing section for removing the developer remaining on the developed sample after development, and a gumming section charged with a gumming solution (a solution obtained by diluting GW-3, produced by Mitsubishi Chemical Co., Ltd., with water by a factor of 2) for protecting the surface of the developed sample.

Time during which the light sensitive planographic printing plate material sample is in contact with the developer was defined as developing time. Thus, the exposed light sensitive planographic printing plate material sample was developed at a developing temperature of 28° C. for 18 seconds in the automatic developing machine above to obtain a planographic printing plate sample.

In the heating section, temperature of the rear surface of the support opposite the light sensitive layer was set to be 115±5° C.

The rear surface temperature was measured employing a thermo label (produced by Nichiyu Giken Co., Ltd.) adhered on the rear surface of the support opposite the light sensitive layer.

(Developer composition 1)

| | |
|---|---|
| Potassium silicate A | 8.0 parts |
| NEWCOL B-13SN (produced | 3.0 parts |
| by Nippon Nyukazai Co., Ltd.) | |
| Ethylenediaminetetraacetic acid disodium | 0.1 parts |
| salt dihydrate | |
| Potassium hydroxide | Amount giving pH 12.45 |

(Evaluation of Planographic Printing Plate Material Sample) Regarding the planographic printing plate sample obtained above, the following evaluation was carried out. The results are shown in Table 3.

### (Sensitivity)

The light sensitive planographic printing plate material sample obtained above was exposed at various exposure energy levels of laser used to form solid images, and developed in the same manner as above to obtain developed solid images. Density of the developed solid images at each exposure energy level was measured employing a densitometer D196 manufactured by GRETAG Co. Ltd.

An exposure energy at which optical density begins decreasing from the saturated solid image density was defined as sensitivity. Sensitivity was represented in terms of energy giving saturated solid image density×0.9.

### (Solubility of Polymerizable Monomer, % by Weight)

Solubility of polymerizable monomer to water or an alkali solution was measured according to the procedure described above.

### (Printing Durability)

The resulting planographic printing plate sample being mounted on a press (DAIYA1F-1 produced by Mitsubishi Jukogyo Co., Ltd.), printing was carried out, wherein a coat paper, printing ink (Toyo King Highecho Magenta M, produced by Toyo Ink Manufacturing Co., Ltd.), and dampening water (SG-51, H solution produced by Tokyo Ink Co., Ltd., Concentration: 1.5%) were used. Every time one thousand prints were printed, a printed sample was taken and evaluated.

The number of prints, which was printed until dots at highlight portions of the sample changed by 3% or filling-up (adhesion of printing ink to non-image portions around dot images) at shadow portions of the sample as compared with a printed sample at initial printing stage, was determined as a measure of printing durability. The more the number is, the higher the printing durability.

### (Developability (Running Property))

Five hundred square meters of each planographic printing plate material sample were processed employing the developing machine and developer each described above, and after processing, sludge produced in the developing tank and sludge adhered on the developed sample were observed.

<Anti-sludge contamination property>

The sludge produced was evaluated according to the following criteria as one measure of anti-contamination property.

A: The sludge was not adhered to the developed sample, which was not problematic.
B: The sludge was adhered to the developed sample in the developing tank, but removed in a washing section from the sample, which was not substantially problematic.
C: The sludge was adhered to the developed sample, causing contamination at non-image portions.

### <Sludge>

After each planographic printing plate material sample was processed in the same manner as above, 100 ml of the developer were placed in a tightly sealed vessel and stored at 55° C. and 20% RH for 7 days. The resulting developer was filtered off to obtain a residue. The residue was dried at 70° C. for one day. The amount of the dried residue was represented in terms of g/liter and evaluated as another measure of anti-contamination property.

The results are shown in Table 3.

TABLE 3

| | | | | | | | | Solubility of | | |
|---|---|---|---|---|---|---|---|---|---|---|
| | Kind of | | | | Print- | | | polymerizable | | |
| Kind of | poly- | | | Sensi- | ing | | | monomer | | |
| polymer | merizable | | Co- | tivity | dura- | Develop- | Sludge | Pure | Alkali | |
| binder | monomer | Initiator | initiator | μj/cm² | bility | ability | g/L | water | solution | Remarks |
| SP-2 | PETA | 1 | 1 | 50 | 20,000 | C | 5 | 0.1 | 0.1 | Comp. |
| SP-2 | PM-2 | 1 | 1 | 20 | 300,000 | A | 1 | 0.9 | 1.1 | Inv. |
| SP-2 | M-12 | 1 | 1 | 15 | 400,000 | A | 0.7 | 1.5 | 1.8 | Inv. |
| SP-2 | M-14 | 1 | 1 | 40 | 100,000 | A | 0.5 | 2.5 | 4 | Inv. |
| SP-2 | M-20 | 1 | 1 | 15 | 400,000 | A | 0.5 | 2 | 2.5 | Inv. |
| SP-2 | M-21 | 1 | 1 | 20 | 300,000 | A | 0.3 | 4 | 8 | Inv. |
| SP-1 | PETA | 1 | 1 | 90 | 5,000 | B | 4 | 0.1 | 0.1 | Comp. |
| SP-1 | PM-2 | 1 | 1 | 20 | 300,000 | A | 0.5 | 0.9 | 1.1 | Inv. |
| SP-1 | M-12 | 1 | 1 | 18 | 350,000 | A | 0.3 | 1.5 | 1.8 | Inv. |
| SP-1 | M-14 | 1 | 1 | 50 | 100,000 | A | 0.2 | 2.5 | 4 | Inv. |
| SP-1 | M-20 | 1 | 1 | 18 | 350,000 | A | 0.2 | 2 | 2.5 | Inv. |
| SP-1 | M-21 | 1 | 1 | 20 | 300,000 | A | 0.1 | 4 | 8 | Inv. |
| SP-2 | PETA | 2 | 2 | 90 | 5,000 | C | 10.0 | 0.1 | 0.1 | Comp. |
| SP-2 | PM-2 | 2 | 2 | 30 | 150,000 | A | 1.4 | 0.9 | 1.1 | Inv. |
| SP-2 | M-12 | 2 | 2 | 30 | 150,000 | A | 1 | 1.5 | 1.8 | Inv. |
| SP-2 | M-14 | 2 | 2 | 15 | 400,000 | A | 0.6 | 2.5 | 4 | Inv. |
| SP-2 | M-20 | 2 | 2 | 20 | 400,000 | A | 0.8 | 2 | 2.5 | Inv. |
| SP-2 | M-21 | 2 | 2 | 20 | 350,000 | A | 0.4 | 4 | 8 | Inv. |

Comp.: Comparative, Inv.: Inventive
Initiator 1 Iron arene compound: IRGACURE261 (produced by Ciba Speciality Chemicals Co. Ltd.)
Initiator 2 Bisimidazole compound: HABI
Co-initiator 1: Tribromoacetyl amide compound
Co-initiator 2: Mercaptobenzoxazole
Initiator 1

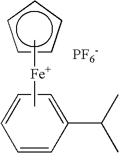

Initiator 2

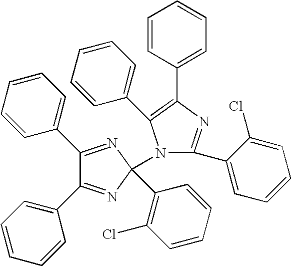

Co-initiator 1

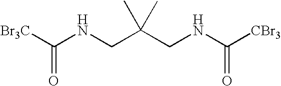

As is apparent from Table 3, inventive samples excel in sensitivity, printing durability, developability, and anti-sludge property, and the polymerizable monomer used in inventive samples excel in solubility.

1-11. (canceled)

12. An ethylenically unsaturated compound having in the molecule a photo-oxidation group and a polymerizable ethylenically unsaturated bond, wherein the ethylenically unsaturated compound dissolves in an amount of not less than 1% by weight in a 25° C. KOH aqueous solution having a pH of 12.5.

13. The ethylenically unsaturated compound of claim 12, wherein the ethylenically unsaturated compound is a polymerizable ethylenically unsaturated compound having in the molecule an amido bond and a secondary or tertiary amino group.

14. The ethylenically unsaturated compound of claim 12, wherein the ethylenically unsaturated compound is a polymerizable ethylenically unsaturated compound further having in the molecule an alkyleneoxy structure.

15. An ethylenically unsaturated compound having in the molecule a photo-oxidation group and a polymerizable ethylenically unsaturated bond, wherein the ethylenically unsaturated compound dissolves in an amount of not less than 1% by weight in a 25° C. pure water.

16. The ethylenically unsaturated compound of claim 15, wherein the ethylenically unsaturated compound is a polymerizable ethylenically unsaturated compound having in the molecule an amido bond and a secondary or tertiary amino group.

17. The ethylenically unsaturated compound of claim 15, wherein the ethylenically unsaturated compound is a polymerizable ethylenically unsaturated compound further having in the molecule an alkyleneoxy structure.

18. A light sensitive composition containing a polymerizable ethylenically unsaturated compound having in the molecule a photo-oxidation group and a polymerizable ethylenically unsaturated bond and a photopolymerization initiator, wherein the ethylenically unsaturated compound dissolves in an amount of not less than 1% by weight in a 25° C. KOH aqueous solution having a pH of 12.5.

19. The light sensitive composition of claim 18, wherein the polymerizable ethylenically unsaturated compound has in the molecule an amido bond and a secondary or tertiary amino group.

20. The light sensitive composition of claim 18, wherein the polymerizable ethylenically unsaturated compound further has in the molecule an alkyleneoxy structure.

21. A light sensitive composition containing a polymerizable ethylenically unsaturated compound having in the molecule a photo-oxidation group and a polymerizable ethylenically unsaturated bond and a photopolymerization initiator, wherein the ethylenically unsaturated compound dissolves in an amount of not less than 1% by weight in a 25° C. pure water.

22. The light sensitive composition of claim 21, wherein the polymerizable ethylenically unsaturated compound has in the molecule an amido bond and a secondary or tertiary amino group.

23. The light sensitive composition of claim 21, wherein the polymerizable ethylenically unsaturated compound further has in the molecule an alkyleneoxy structure.

24. A light sensitive planographic printing plate material comprising a support and provided thereon, a light sensitive layer containing a polymerizable ethylenically unsaturated compound having in the molecule a photo-oxidation group and a polymerizable ethylenically unsaturated bond, a photopolymerization initiator and a polymer binder, wherein the ethylenically unsaturated compound dissolves in an amount of not less than 1% by weight in a 25° C. KOH aqueous solution having a pH of 12.5.

25. The light sensitive planographic printing plate material of claim 24, wherein the light sensitive layer further contains an infrared absorbing agent, and wherein the photopolymerization initiator is a polyhalogen compound and the polymer binder is a water-soluble polymer binder.

26. A light sensitive planographic printing plate material comprising a support and provided thereon, a light sensitive layer containing a polymerizable ethylenically unsaturated compound, a photopolymerization initiator, a polymer binder, and an infrared absorbing agent, wherein the polymerizable ethylenically unsaturated compound is a polymerizable ethylenically unsaturated compound having in the molecule a photo-oxidation group, the photopolymerization initiator is a polyhalogen compound, and the polymer binder is a water-soluble polymer binder.

27. The light sensitive planographic printing plate material of claim 26, wherein the polymerizable ethylenically unsaturated compound dissolves in an amount of not less than 1% by weight in a 25° C. pure water.

28. A light sensitive planographic printing plate material comprising a support and provided thereon, a light sensitive layer containing a polymerizable ethylenically unsaturated compound, a photopolymerization initiator, a polymer binder, and an infrared absorbing agent, wherein the polymerizable ethylenically unsaturated compound is a polymerizable ethylenically unsaturated compound having in the molecule an amido bond and a secondary or tertiary amino group.

29. The light sensitive planographic printing plate material of claim 28, wherein the polymerizable ethylenically unsaturated compound dissolves in an amount of not less than 1% by weight in a 25° C. pure water.

30. A printing process comprising the steps of mounting a light sensitive planographic printing plate material without carrying out development, followed by printing, wherein the light sensitive planographic printing plate material comprises a support and provided thereon, a light sensitive layer containing a polymerizable ethylenically unsaturated compound having in the molecule a photo-oxidation group and a polymerizable
